(19) United States
(12) Patent Application Publication
(10) Pub. No.: US 2013/0177979 A1
(43) Pub. Date: Jul. 11, 2013

(54) METHODS AND COMPOSITIONS FOR CELL PERMEABLE STAT3 INHIBITOR

(71) Applicant: James Turkson, Orlando, FL (US)

(72) Inventor: James Turkson, Orlando, FL (US)

(73) Assignee: University of Central Florida Research Foundation, Inc., Orlando, FL (US)

(21) Appl. No.: 13/806,639

(22) PCT Filed: Jun. 22, 2011
(86) PCT No.: PCT/US2011/041537

§ 371 Date: Mar. 25, 2013

Related U.S. Application Data
(60) Provisional application No. 61/357,134, filed on Jun. 22, 2010.

(51) Int. Cl. C07K 14/435

(52) U.S. Cl. CPC C07K 14/435
USPC 435/348; 530/324; 530/326; 536/23.5; 435/320.1; 435/252.3; 435/254.2; 435/325

(57) ABSTRACT

Disclosed herein are compositions for inhibition of Stat, particularly Stat3. Disclosed herein are compositions comprising cell permeable Stat3 inhibitors. Compositions may comprise peptides, polypeptides, antibodies, nucleic acids, vectors, and host cells for making, using, assaying, and evaluating Stat3 inhibitors. Disclosed herein are methods for making and using the disclosed compositions.

### CROSS REFERENCE TO RELATED PATENT APPLICATIONS

This application claims priority to U.S. Provisional Application Ser. No. 61/357,134 filed Jun. 22, 2010, which is herein incorporated in its entirety.

### STATEMENT REGARDING FEDERALLY SPONSORED RESEARCH OR DEVELOPMENT

This invention was made with government support under National Cancer Institute grant numbers CA106439 and CA128865. The government has certain rights in the invention.

### TECHNICAL FIELD

This invention relates to the field of molecular biology and protein biology including polypeptides and peptidomimetics. This application also relates to the fields of cell signaling, tumor biology, and cancer.

### BACKGROUND OF THE INVENTION

Knowledge of the molecular basis of cancer potentially expands the number of strategies to target cancer cells for therapy. Multiple genetic alterations in cancer frequently result in aberrations in the biochemical properties of signaling molecules, leading to dysregulation of signal transduction mechanisms in tumors and consequently malignant progression.

The Stat (Signal Transcription and Activation of Transcription) proteins are a family of cytoplasmic proteins important in cell proliferation, differentiation, apoptosis and survival. Stat activated dimers have been shown to interact with specific promoter regulatory elements to induce target gene transcription. Stats are triggered through extracellular cytokine and growth factor stimulation resulting in receptor dimerization and activation. Phosphorylation of a tyrosine residue provides binding sites for the recruitment of monomeric, non-phosphorylated Stat proteins via their Src homology 2 (SH2) domain. Receptor-bound Stat3 is then tyrosine phosphorylated by receptor and/or nonreceptor tyrosine kinases such as Src and JAK. Phosphorylated Stat proteins are then released from the receptor, and dimerization occurs through reciprocal phosphotyrosine-SH2 interaction. Stat dimers translocate to the nucleus and bind with promoter regulatory elements.

In normal functioning cells, Stat activation is transitory and tightly regulated. However, aberrant Stat activation leads to the up-regulation of oncogenic pathways through dysregulated growth, suppression, angiogenesis and survival. Studies have established a link between Stat3 and the growth and survival of transformed and tumor cells. In a number of human solid and hematological tumors, studies have identified a high frequency of abnormal activation of Stat3. In many tumor cells harboring persistent Stat3 activity, inhibition of Stat3 signaling induces growth arrest and apoptosis.

What is needed are methods and compositions for inhibiting Stat activation and for providing treatments for dysregulated growth, suppression, angiogenesis and cellular survival conditions in humans and other organisms that have Stat or Stat-like activation.

### SUMMARY

The present invention comprises methods and compositions for inhibition of Stat, particularly Stat3. Disclosed herein are compositions for cell permeable Stat3 inhibitors. Compositions may comprise peptides, polypeptides, antibodies, nucleic acids, vectors, and host cells for making, using, assaying, and evaluating Stat3 inhibitors.

Methods of the present invention comprise methods for inhibiting Stat or Stat activation, and inhibiting the growth or replication of a cell having abnormal growth or replication or whose growth or replication is uncontrolled, such as a cancer cell. Methods may comprise inhibiting Stat function by contacting a cell expressing a Stat with a cell permeable peptidomimetic of the invention wherein the peptidomimetic inhibits Stat or Stat3.

### FIGURES

FIG. 1A-G are graphs that show the results of the surface plasmon resonance (SPR) analysis of the binding of SPI, Stat3 SH2 domain, and full-length Stat3 for (A) GpYLPQTV, (B) PpYLKTK, (C) GpYIKTE, (D) GpYVKPQ, (E) PEpYINQS, (F) PVpYHNQP, and (G) S3I-201.

FIG. 2 A-E are graphs that show the results of the fluorescence polarization (FP) assay of the binding to the 5-carboxyfluorescein-GpYLPQTV-NH2 probe of (A) increasing concentration of purified His-Stat3; (B) increasing concentrations of SPI; (C) a fixed amount of purified His-Stat3 (200 nM) in the presence of increasing concentrations of S31-201; (D) a fixed amount of SPI in the presence of increasing concentrations of S31-201; and (E) a fixed amount of Stat3 in the presence of increasing concentrations of SPI.

FIG. 3 A-C show graphs of cytosolic extracts of total protein from 24-hour SPI-treated or untreated (A) NIH3T3/v-Src/pLucTKS3 fibroblasts, (B) NIH3T3/v-Src/pLucSRE fibroblasts, and (C) NIH3T3/hEGFR fibroblasts; FIG. 3D show gels of nuclear extracts of total protein from malignant cells treated for 24 hours with or without SPI and subjected to in vitro DNA-binding assay using the radiolabeled hSIE that binds Stat1 and Stat3 and analyzed by EMSA; FIG. 3E shows gels of nuclear extracts of total protein from (E) EGF-stimulated NIH3t3/hEGFR cells treated for 24 hours with or without SPI and subjected to in vitro DNA-binding assay using the radiolabeled hSIE that binds Stat1 and Stat3 and the MGFe probe that binds Stat1 and Stat5 and analyzed by EMSA; FIG. 3F-G shows SDS-Page and Western blotting analyses of whole cell lysates of protein prepared from SPI-treated or untreated NIH3T3/v-Src and MDA-MB-231 cells and probing for pY705Stat3 (top panel in (F)), Stat3 (top panel in (F)), pErk 1/2 (bottom panel in (F)), and Erk 1/2 (bottom panel in (F)), and SPI-treated or untreated NIH3T3/v-Src cells probing for general pTyr profile (G).

FIG. 4 is a gel showing an in vitro DNA-binding assay showing nuclear extracts prepared from MDA-MB-231 cells pre-treated with or without SPI prior to treatment with or without sodium orthovanadate and subjected to in vitro DNA-binding assay using the radiolabeled hSIE probe and analyzed by EMSA.

FIG. 5 A-C show (A) a graph showing viability of cells untreated or treated once with increasing concentration of SPI; flow cytometry analysis of human breast cancer cells or normal mouse fibroblasts following no treatment or treatment with SPI and binding to (B) Annexin V and (C) 7-AAD.

FIG. 6 A-C shows (A) comparison of tumor volume size between control (DMSO-treated) mice and mice treated with SPI, (B) DNA-binding activity/EMSA analysis of tumor and control lysates using hSIE probe; and (C) Western blotting analysis of lysates from control tumor and residual tumors from SPI-treated mice.

FIG. 7 shows nuclear extracts of equal total protein that were treated with or without the polypeptide comprising SEQ ID NO:21 and subjected to in vitro DNA-binding assay using the radiolabeled hSIE probe.

### DETAILED DESCRIPTION

The present invention comprises methods and compositions for inhibition of Signal Transducer and Activator of Transcription (Stat) proteins. A composition of the present invention comprises a Stat3 inhibitor, a 28-mer peptide, referred to herein as SPI, derived from the Stat3 SH2 domain, which mimics Stat3 functional and biochemical properties. SPI and Stat3 (or Stat3 SH2 domain) bind with similar affinities to known Stat3-binding phosphotyrosine (pTyr) peptide motifs, including those of the epidermal growth factor receptor (EGFR) and the high-affinity, IL-6R/130-derived pY-peptide, GpYLPQTV-NH₂. Compositions of the present invention comprising SPI function as a potent and selective inhibitors of Stat3 SH2 domain:pTyr interactions and disrupt the binding of Stat3 to the IL-6R/gpl30 peptide, GpYLPQTV-NH₂.

Fluorescence imaging and immunofluorescence staining/laser-scanning confocal microscopy showed SPI was cell membrane-permeable, associated with the cytoplasmic tail of EGFR in NIH3T3/hEGFR, and was present in the cytoplasm. SPI was localized at the plasma membrane and in the nucleus in malignant cells harboring persistently-active Stat3. Compositions comprising SPI specifically blocked constitutive Stat3 phosphorylation, DNA-binding activity, and transcriptional function in malignant cells. Currently, though not wishing to be bound by any particular theory, it is believed that SPI compositions have little or no effect on the induction of Stat1, Stat5, and Erk1/2^{MAPK} pathways, or on general pTyr profile at the concentrations of SPI that inhibit Stat3 activity. Treatment with SPI of human breast, pancreatic, prostate, and non-small cell lung cancer cells harboring constitutively-active Stat3 induced extensive morphology changes that are associated with viability loss and apoptosis. The present invention comprises compositions of SPI and methods of use of such compositions for uses including molecular probes for interrogating Stat3 signaling and functioning as a selective inhibitor of Stat3 activation with antitumor cell effects, in vivo, in vitro and in silico.

It is currently believed that the binding of cytokines or growth factors to cognate receptors initiates a cascade of molecular events that culminate in the activation of the Signal Transducer and Activator of Transcription (Stat) family of proteins (Bromberg 2000; Darnell 2002). Among these is the recruitment of Stats, via the SH2 domain, to the receptor phosphotyrosine (pTyr) peptide motifs, which brings them into close proximity for phosphorylation on a tyrosyl residue by growth factor receptor tyrosine kinases, Janus kinases (Jaks), and the Src family kinases. Consequently, dimerization between two Stat monomers is promoted through a reciprocal pTyr-SH2 domain interaction, and the active Stat dimers in the nucleus bind to specific DNA-response elements in the promoters of target genes and regulate gene expression. In response to growth factors and cytokines, normal Stat signaling promotes cell growth and differentiation, development, inflammation and immune responses.

The Stat proteins are modular in structure and contain N-terminal domain. Coiled-coil domain, DNA-binding domain, SH2 domain, and a Transcriptional activation domain, with each domain engaging in molecular events for promoting Stat functions. In particular, the SH2 domain mediates interactions with specific pTyr peptide motifs, including promoting the association with receptors and holding of two activated Stat monomers together in a reciprocal SH2 domain:pTyr interactions in Stat:Stat dimerization. Among the Stat family members, Stat3 and Stat5 have been implicated in malignant transformation and tumorigenesis (Yu et al., 2004; Yue et al., 2008; Turkson 2004; Turkson et al., 2000; Darnell 2005) and have become valid targets for anticancer drug design.

Prior to the present invention, the focus of the existing Stat3 drug discovery efforts have been on disrupting the Stat3 SH2 domain:pTyr peptide interactions and the approaches have largely been directed at SH2 domain antagonists, which are pTyr peptide mimics that compete for the binding to the Stat3 SH2 domain (Turkson et al., 2008; Turkson 2004; Fletcher et al., 2008). One of the major limitations of this approach has been finding a membrane-permeable, optimum pTyr substitute that retains the high binding affinities of the native pTyr peptide motifs against which these antagonists will be competing for the binding to the Stat3 SH2 domain. The present invention comprises compositions and methods for a Stat3 SH2 domain-mimic.

Structural information from the computational modeling of the native pTyr peptide, PpYLKTK bound to the Stat3 SH2 domain, per the crystal structure of Stat3β (Becker et al., 1998) aided in the design of a 28-mer peptide, SEQ ID. NO. 1, referred to herein as SPI, from the Stat3 SH2 domain. SPI (Stat Protein Inhibitor) and variants thereof retain the binding characteristics of the SH2 domain. In vitro biochemical and biophysical studies indicated SPI, like Stat3 bound to cognate pTyr-peptide motifs with a similar affinity. Compositions of the present invention comprising SPI or SPI variants blocked the binding of Stat3 (or Stat3 SH2 domain) to cognate pTyr peptide motifs, and function as selective inhibitors of constitutive Stat3 activation in human breast, prostate, pancreatic, and non-small cell lung cancer cells, with antitumor cell effects.

The present invention comprises compositions comprising a Stat3 SH2 domain mimicking polypeptide comprising SEQ ID NO. 1. SEQ ID 1 is FISKERERAILSTKPPGTFLLRFSESSK. SPI, described by SEQ ID NO:1, or its variants, described by other SEQ ID NOs disclosed herein, may be made by any method known for producing polypeptides. For example, the present invention comprises a Stat3 SH2 domain mimicking polypeptide that is a recombinant polypeptide. The present invention comprises a Stat3 SH2 domain mimicking polypeptide that is a synthetic polypeptide. The present invention comprises a Stat3 SH2 domain mimicking polypeptide that has binding and/or biochemical characteristics of some or all of Stat3 or Stat3 SH2 domain. Polypeptides disclosed herein that have binding characteristics similar to those of Stat3 SH2 domain are referred to herein as Stat3 SH2 domain mimicking polypeptides. As used herein, a mimicking polypeptide is a molecule made from amino acids that has the same or a similar binding response, such as to antibodies, peptides, nucleic acids, or small molecules, as does the protein being mimicked, and/or has the same or similar biochemical responses, such as functioning in the same or similar pathways, or acted on or acts with, the same or similar proteins, nucleic acids, receptors, or cellular components as does the protein being mimicked.

Disclosed herein are inhibitors of Stat3. In an aspect, the Stat3 inhibitor is a polypeptide. Stat3 inhibitors disclosed herein can be recombinant or synthetic polypeptides. Polypeptides may comprise SEQ ID NO:1, or portions thereof. In an aspect, a disclosed Stat3 inhibitor is FISKERERAILSTKPPGTFLLRFSESSK. In an aspect, a polypeptide can comprise SEQ ID NO:21. In an aspect, a disclosed Stat3 inhibitor is ISKERERAILSTKPP.

Disclosed herein are Stat3 SH2 domain mimicking polypeptides. Stat3 SH2 domain mimicking polypeptides mimic or replicate Stat3 biochemical properties. In an aspect, a disclosed polypeptide comprises SEQ ID NO:1. SEQ ID NO:1 is FISKERERAILSTKPPGTFLLRFSESSK.

Disclosed herein is SEQ ID NO:2, which represents amino acids 1-770 of Stat3 of Mus musculus (the 28 amino acids corresponding SEQ ID NO:1 are underlined). SEQ ID NO: 2 is MAQWNQLQQL DTRYLKQLHQ LYSDTFPMEL RQFLAPWIES QDWAYAASKE SHATLVFHNL LGEIDQQYSR FLQESNVLYQ HNLRRIKQFL QSRYLEKPME IARIVARCLW EESRLLQTAA TAAQQGGQAN HPTAAVVTEK QQMLEQHLQD VRKRVQDLEQ KMKVVENLQD DFDFNYKTLK SQGDMQDLNG NNQSVTRQKM QQLEQMLTAL DQMRRSIVSE LAGLLSAMEY VQKTLTDEEL ADWKRRQQIA CIGGPPNICL DRLENWITSL AESQLQTRQQ IKKLEELQQK VSYKGDPIVQ HRPMLEERIV ELFRNLMKSA FVVERQPCMP MHPDRPLVIK TGVQFTTKVR LLVKFPELNY QLKIKVCIDK DSGDVAALRG SRKFNILGTN TKVMNMEESN NGSLSAEFKH LTLREQRCGN GGRANCDASL IVTEELHLIT FETEVYHQGL KIDLETHSLP VVVISNICQM PNAWASILWY NMLTNNPKNV NFFTKPPIGT WDQVAEVLSW QFSSTTKRGL SIEQLTTLAE KLLGPGVNYS GCQITWAKFC KENMAGKGFS FWVWLDNIID LVKKYILALW NEGYIMGFIS KERERAILST KPPGTFLLRF SESSKEGGVT FTWVEKDISG KTQIQSVEPY TKQQLNNMSF AEIIMGYKIM DATNILVSPL VYLYPDIPKE EAFGKYCRPE SQEHPEADPG SAAPYLKTKF ICVTPTTCSN TIDLPMSPRT LDSLMQFGNN GEGAEPSAGG QFESLTFDMD LTSECATSPM.

Disclosed herein is SEQ ID NO:3, which represents amino acids 1-770 of human Stat3 (the 28 amino acids corresponding to the sequence of SEQ ID NO:1). SEQ ID NO:3 is MAQWNQLQQL DTRYLEQLHQ LYSDSFPMEL RQFLAPWIES QDWAYAASKE SHATLVFHNL LGEIDQQYSR FLQESNVLYQ HNLRRIKQFL QSRYLEKPME IARIVARCLW EESRLLQTAA TAAQQGGQAN HPTAAVVTEK QQMLEQHLQD VRKRVQDLEQ KMKVVENLQD DFDFNYKTLK SQGDMQDLNG NNQSVTRQKM QQLEQMLTAL DQMRRSIVSE LAGLLSAMEY VQKTLTDEEL ADWKRRQQIA CIGGPPNICL DRLENWITSL AESQLQTRQQ IKKLEELQQK VSYKGDPIVQ HRPMLEERIV ELFRNLMKSA FVVERQPCMP MHPDRPLVIK TGVQFTTKVR LLVKFPELNY QLKIKVCIDK DSGDVAALRG SRKFNILGTN TKVMNMEESN NGSLSAEFKH LTLREQRCGN GGRANCDASL IVTEELHLIT FETEVYHQGL KIDLETHSLP VVVISNICQM PNAWASILWY NMLTNNPKNV NFFTKPPIGT WDQVAEVLSW QFSSTTKRGL SIEQLTTLAE KLLGPGVNYS GCQITWAKFC KENMAGKGFS FWVWLDNIID LVKKYILALW NEGYIMGFIS KERERAILST KPPGTFLLRF SESSKEGGVT FTWVEKDISG KTQIQSVEPY TKQQLNNMSF AEIIMGYKIM DATNILVSPL VYLYPDIPKE EAFGKYCRPE SQEHPEADPG SAAPYLKTKF ICVTPTTCSN TIDLPMSPRT LDSLMQFGNN GEGAEPSAGG QFESLTFDME LTSECATSPM.

A variant of SEQ ID NO:1 is SEQ ID NO:4, which is represented by the amino acid sequence FISKERERAILSPKPPGTFLLRFSESSK.

The present invention comprises polypeptides that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to SEQ ID NO:1. Disclosed herein are polypeptide variants that have 70-74, 75-79, 80-84, 85-89, 90-94, 95-99 percent homology to SEQ ID NO:1. Disclosed polypeptide variants comprise 70-99, 75-95, or 80-90 percent homology to SEQ ID NO:1, or 70-80, 80-90, or 90-100 percent homology to SEQ ID NO:1.

An aspect of the present invention comprises a polypeptide comprising SEQ ID NO:1 and additional amino acids on the amino end of SEQ ID NO:1, or on the carboxy end of SEQ ID NO:1, or on both the amino end and the carboxy end of SEQ ID NO:1. For example, disclosed herein is a polypeptide comprising, for example, 20 (e.g., SEQ ID NO:5), 19, 18, 17, 16, 15 (e.g., SEQ ID NO:6), 14, 13, 12, 11, 10 (e.g., SEQ ID NO:7), 9, 8, 7, 6, 5 (e.g., SEQ ID NO:8), 4, 3, 2, or 1 (e.g., SEQ ID NO:7) additional amino acids that are on amino end of SEQ ID NO:1, and further disclosed is a polypeptide comprising, for example, 20 (e.g., SEQ ID NO:10), 19, 18, 17, 16, 15 (e.g., SEQ ID NO:11), 14, 13, 12, 11, 10 (e.g., SEQ ID NO:12), 9, 8, 7, 6, 5 (e.g., SEQ ID NO:13), 4, 3, 2, or 1 (e.g., SEQ ID NO:14) additional amino acids that are on the carboxy end of SEQ ID NO:1, or a combination of 20, 19, 18, 17, 16, 15 (e.g., SEQ ID NO:15), 14, 13, 12, 11 10, 9, 8, 7 (e.g., SEQ ID NO:16), 6, 5 (e.g., SEQ ID NO:17), 4, 3, 2, or 1 (e.g., SEQ ID NO:17) additional amino acids, or a combination thereof, that are on both the amino and carboxy ends of SEQ ID NO:1 (e.g., SEQ ID NOs:15-18).

An aspect of the present invention comprises a polypeptide comprising SEQ ID NO:1 and additional amino acids on the amino end of SEQ ID NO:1, or on the carboxy end of SEQ ID NO:1, or on both the amino end and the carboxy end of SEQ ID NO:1. For example, disclosed herein is a polypeptide comprising, for example, 15-20 (e.g., SEQ ID NOs:5 and 6), or 10-15 (e.g., SEQ ID NOs:6 and 7), or 5-10 (e.g., SEQ ID NOs:7 and 8), or 1-5 (e.g., SEQ ID NOs:8 and 9) additional amino acids that are on the amino end of SEQ ID NO:1, and further disclosed is a polypeptide comprising, for example, 15-20 (e.g., SEQ ID NOs:10 and 11), or 10-15 (e.g., SEQ ID NOs:11 and 12), or 5-10 (e.g., SEQ ID NOs:12 and 13), or 1-5 (e.g., SEQ ID NOs:13 and 14) additional amino acids that are on the carboxy end of SEQ ID NO:1, or a combination of 15-20, or 10-15, or 5-10, or 1-5 additional amino acids, or a combination thereof, that are on both the amino and carboxy ends of SEQ ID NO:1.

Disclosed herein are Stat3 SH2 domain mimicking polypeptides that mimic or replicate Stat3 biochemical properties. In an aspect, the disclosed polypeptide comprises SEQ ID NO:21. SEQ ID NO:21 is ISKERERAILSTKPP.

An aspect of the present invention comprises a polypeptide comprising a portion of SEQ ID NO:1, for example, SEQ ID NO:21. Disclosed is SEQ ID NO:22, which represents amino acids 591 to 613 of SEQ ID NO:3. SEQ ID NO:22 is KERERAILSTKPPGTFLLRFSES.

Exemplary variant polypeptides comprising SEQ ID NO:1 are disclosed in Table 1. The 28-mer of SEQ ID NO:1 is underlined in each of the exemplary variant polypeptides.

TABLE 1
Exemplary Variant Polypeptides Comprising SEQ ID NO: 1

| AMINO ACID SEQUENCE | SEQ ID NO: |
|---|---|
| IIDLVKKYILALWNEGYIMGFISKERERAILSTKPPGTFLLRFSESSK | NO: 5 |
| KKYILALWNEGYIMGFISKERERAILSTKPPGTFLLRFSESSK | NO: 6 |
| ALWNEGYIMGFISKERERAILSTKPPGTFLLRFSESSK | NO: 7 |
| GYIMGFISKERERAILSTKPPGTFLLRFSESSK | NO: 8 |
| GFISKERERAILSTKPPGTFLLRFSESSK | NO: 9 |
| FISKERERAILSPKPPGTFLLRFSESSKEGGITFTWVEKDINGKTQIQ | NO: 10 |
| FISKERERAILSPKPPGTFLLRFSESSKEGGITFTWVEKDING | NO: 11 |
| FISKERERAILSPKPPGTFLLRFSESSKEGGITFTWVE | NO: 12 |
| FISKERERAILSPKPPGTFLLRFSESSKEGGIT | NO: 13 |
| FISKERERAILSPKPPGTFLLRFSESSKE | NO: 14 |
| ALWNEGYIMGFISKERERAILSTKPPGTFLLRFSESSKEGGVTFTWVE | NO: 15 |
| NEGYIMGFISKERERAILSTKPPGTFLLRFSESSKEGGVTFT | NO: 16 |
| GYIMGFISKERERAILSTKPPGTFLLRFSESSKEGGVT | NO: 17 |
| GFISKERERAILSTKPPGTFLLRFSESSKE | NO: 18 |

Disclosed herein are polypeptides that are homologous to polypeptides comprising SEQ ID NO:1. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their peptide or nucleic acid sequences. Many of the methods for determining homology between two evolutionarily related molecules are routinely applied to any two or more nucleic acids or proteins for the purpose of measuring sequence similarity regardless of whether they are evolutionarily related or not. Thus, the polypeptides disclosed herein comprise polypeptides of multiple species, including but not limited to mouse, human, chicken, pig, rat, cow, chimpanzee, zebrafish, etc. Further, the disclosed Stat3 SH2 domains comprise domains from multiple species, including but not limited to mouse, human, chicken, pig, rat, cow, chimpanzee, zebrafish, etc.

Polypeptides disclosed herein encompass naturally occurring or synthetic molecule, and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide.

Disclosed herein are multimers of one or more polypeptides disclosed herein. In an aspect, a multimer comprises more than one of the monomers disclosed herein. A disclosed multimer can be a 56-mer, an 84-mer, a 112-mer, a 140-mer, a 168-mer, or the like. For example, in an aspect, the monomer comprises a sequence of 28 amino acids, such as the 28 amino acids of SEQ ID NO:1. In an aspect, the monomer comprise a variant of SEQ ID NO:1, such as, for example, the sequence of SEQ ID NO:4. A disclosed multimer can be a 30-mer, an 45-mer, a 60-mer, a 75-mer, a 90-mer, or the like. In an aspect, the disclosed monomer comprises a sequence of 15 amino acids, such as the 15 amino acids of SEQ ID NO:21. In an aspect, the disclosed multimers comprise a combination of one or more monomers comprising SEQ ID NO:1 and one or more monomers comprising a variant of SEQ ID NO:1. In an aspect, the disclosed multimers comprise a combination of one or more monomers comprising SEQ ID NO:21 and one or more monomers comprising a variant of SEQ ID NO:21. Disclosed are compositions comprising the disclosed multimers, including compositions comprising monomers comprising the amino acid sequence of SEQ ID NO:1. Disclosed are compositions comprising the disclosed multimers, including compositions comprising monomers comprising the amino acid sequence of SEQ ID NO:21.

Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation.

Also, polypeptides disclosed herein can have one or more types of modifications. Numerous variants or derivatives of the peptides and analogs of the invention are also contemplated. As used herein, the term “analog” is used interchangeably with “variant” and “derivative.” Variants and derivatives are well understood to those of skill in the art and can involve amino acid sequence modifications. Such amino acid sequence modifications typically fall into one or more of three classes: substitutional; insertional; or deletional variants. Insertions include amino and/or carboxyl terminal fusions as well as intrasequence insertions of single or multiple amino acid residues. Insertions ordinarily are smaller insertions than those of amino or carboxyl terminal fusions, for example, on the order of one to four residues. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Substitutions, deletions, insertions or any combination thereof may be combined to arrive at a final derivative or analog.

The polypeptides disclosed herein can comprise one or more substitutional variants, i.e., a polypeptide in which at least one residue has been removed and a different residue inserted in its place. Such substitutions generally are made in accordance with Table 2 and are referred to as conservative substitutions.

TABLE 2
Exemplary Conservative Amino Acid Substitutions

| Original | Exemplary Conservative |
|---|---|
| Residue | Substitutions |
| Ala | Ser |
| Arg | Gly, Gln |
| Asn | Gln; His |
| Asp | Glu |
| Cys | Ser |
| Gln | Asn, Lys |
| Glu | Asp |
| Gly | Ala |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions that are generally expected to produce the greatest changes in the protein properties are those in which: (a) the hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or hystidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, or (e) by increasing the number of sites for sulfation and/or glycosylation.

Polypeptides of the present invention are produced by any method known in the art. One method of producing the disclosed polypeptides is to link two or more amino acid residues, peptides or polypeptides together by protein chemistry techniques. For example, peptides or polypeptides are chemically synthesized using currently available laboratory equipment using either Fmoc (9-fluorenylmethyloxycarbonyl) or Boc (tert-butyloxycarbonoyl) chemistry. A peptide or polypeptide can be synthesized and not cleaved from its synthesis resin, whereas the other fragment of a peptide or protein can be synthesized and subsequently cleaved from the resin, thereby exposing a terminal group, which is functionally blocked on the other fragment. By peptide condensation reactions, these two fragments can be covalently joined via a peptide bond at their carboxyl and amino termini, respectively. Alternatively, the peptide or polypeptide is independently synthesized in vivo. Once isolated, these independent peptides or polypeptides may be linked to form a peptide or fragment thereof via similar peptide condensation reactions.

For example, enzymatic ligation of cloned or synthetic peptide segments allow peptide fragments to be joined to produce larger peptide fragments, polypeptides or whole protein domains. Alternatively, native chemical ligation of synthetic peptides can be utilized to synthetically construct larger peptides or polypeptides from shorter peptide fragments. This method consists of a two-step chemical reaction. The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolim et al., 1992; Clark-Lewis et al., 1994; Clark-Lewis et al., 1991; Rajarathnam et al., 1994).

Alternatively, unprotected peptide segments are chemically linked where the bond formed between the peptide segments as a result of the chemical ligation is an unnatural (non-peptide) bond (Schnolzer et al., 1992). This technique has been used to synthesize analogs of protein domains as well as large amounts of relatively pure proteins with full biological activity (deLisle et al., 1992).

Also disclosed are methods for generating the disclosed peptides and polypeptides in vivo. For example, in an aspect, the disclosed peptides of the present invention are translation products of nucleic acids. In an aspect, nucleic acids are introduced into cells, and the cells express nucleic acids, which are translated to form the disclosed peptides. The present invention also provides for a host cell comprising a nucleic acid encoding one or more of the disclosed peptides. In an aspect, bacterial, yeast, Dictyostelium discoideum, insect, and mammalian cell expression systems can be used to produce the peptides of the present invention. The disclosed peptides can be used as human and animal therapeutics. The art is familiar with expression systems that produce, in an efficient and inexpensive manner, large quantities of soluble, desirable peptide products.

Such an expression system comprises host cells, which can be eukaryotic cells or prokaryotic cells. In the case of eukaryotic cells, retrovirus or adenovirus based vectors can be used to put the nucleic acid or the invention into the host cell. Methods known to one of skill in the art to insert the nucleic acids or polypeptides in host cells are encompassed within this invention. The following are non-limiting examples of such methods: naked DNA transfection, lipofectin-mediated transfer, transformation, micro-injection of nucleic acid into a cell, or calcium-phosphate precipitation tranfection methods.

Host cells can be obtained from commercial sources such as the American Type Culture Collection (ATCC). Host cells can be grown in liquid media culture or on tissue culture plates. The growth conditions will be dependent upon the specific host cells used and such conditions would be known to one of skill in the art. Transfection and growth of host cells is described in Maniatis et al. The invention provides for a recombinant cell expressing a heterologous or homologous nucleic acid encoding the peptide of the claimed invention. The invention also provides for host cell producing a recombinant polypeptide of the invention.

Disclosed peptides generated during in vivo cultivation can be easily collected using conventional purification and separation techniques, such as salting out, dialysis, filtration, centrifugation, concentration and lypholization. If a further purified peptide preparation is desirable, then a peptide preparation of the highest purity can be obtained by the above mentioned techniques in combination with more sensitive conventional purification and separation techniques, such as adsorption and desorption with ion exchange resin, gel filtration, affinity chromatography, isoelectric point fractionation, electrophoresis, etc.

The present invention comprises methods and compositions comprising a polypeptide that binds to receptor phosphotryosine peptide motifs (pTyr). In an aspect, the pTyr motif is a motif of the epidermal growth factor receptor. In an aspect, the pTyr motif is a motif of the IL-6 receptor. A polypeptide that binds to a receptor phosphotryosine peptide motif comprises SEQ ID NO:1. A polypeptide that binds to a receptor phosphotryosine peptide motif comprises SEQ ID NO:21. A polypeptide that binds to a receptor phosphotryosine peptide motif comprises one or more of the polypeptides disclosed herein.

The present invention comprises methods and compositions comprising polypeptides that modulate binding of cognate pTyr peptides to Stat3. In an aspect, disclosed polypeptides inhibit binding of entities, including but not limited to, small molecules or polypeptides, to the SH2 domain of a Stat protein, for example, Stat3 SH2 domain. In an aspect, disclosed polypeptides inhibit binding of Stat3 or the Stat3 SH2 domain with one or more of the following: native pTyr peptide, PpYLKTK, native IL-6R/gp-130 derived peptide, GpYLPQTV-NH2, the Stat3 phosphopeptide, pY705Stat3, and the EGFR motif pY1068EGFR.

The present invention comprises methods and compositions comprising polypeptides that modulate phosphorylation of a Stat monomer. The present invention comprises methods and compositions comprising polypeptides that inhibit phosphorylation of a Stat3 monomer. In an aspect, the disclosed polypeptides inhibit phosphorylation of a Stat monomer, such as Stat3 monomer, wherein phosphorylation is accomplished by at least one of growth factor receptor tyrosine kinases, Janus kinases (Jaks), and/or the Src family kinases.

The present invention comprises a polypeptide that modulate dimerization of two Stat monomors. In an aspect, the dimerization of Stat3 monomers is inhibited by disclosed polypeptides. In an aspect, disclosed polypeptides inhibit dimerization of Stat monomers wherein at least one of the monomers is Stat3. In an aspect, disclosed polypeptides inhibit the dimerization of Stat monomers wherein at least one of the monomers is Stat5. In an aspect, disclosed polypeptides inhibit dimerization of Stat monomers wherein at least one of the monomers is not Stat1.

The present invention comprises methods and compositions comprising a polypeptide that competes with binding sites for Stat3. An example of a polypeptide of the present invention is SPI, SEQ ID NO:1. An example of a polypeptide of the present invention is SEQ ID NO:21. An example of a polypeptide of the present invention comprises SPI, SEQ ID NO:1, or one or more of the disclosed polypeptide and sequences herein. In an aspect, a disclosed polypeptide may inhibit phosphorylation of cellular Stat3, synthetic Stat3, constitutively expressed Stat3, Stat monomers, Stat3 monomers, and/or Stat3 SH2 domain, in vivo, in vitro and/or in silico. In an aspect, a polypeptide of the present invention may inhibit DNA binding by cellular Stat3, synthetic Stat3, constitutively expressed Stat3, Stat monomers, Stat3 monomers, and/or Stat3 SH2 domain, in vivo, in vitro and/or in silico. In an aspect, a polypeptide of the present invention may inhibit transcription activities by cellular Stat3, synthetic Stat3, constitutively expressed Stat3, Stat monomers, Stat3 monomers, and/or Stat3 SH2 domain, in vivo, in vitro and/or in silico. In an aspect, a polypeptide of the present invention may inhibit the activities of cellular Stat3, synthetic Stat3, constitutively expressed Stat3, Stat monomers, Stat3 monomers, and/or Stat3 SH2 domain, in vivo, in vitro and/or in silico.

The present invention comprises methods and compositions comprising a peptidomimetic that competes with binding sites for Stat3. An example of a peptidomimetic of the present invention comprises SPI, SEQ ID NO:1. An example of a peptidomimetic of the present invention comprises SEQ ID NO:21. An example of a peptidomimetic of the present invention is one or more of the disclosed polypeptide and sequences herein. In an aspect, a disclosed peptidomimetic may inhibit phosphorylation of cellular Stat3, synthetic Stat3, constitutively expressed Stat3, Stat monomers, Stat3 monomers, and/or Stat3 SH2 domain, in vivo, in vitro and/or in silico. In an aspect, a peptidomimetic of the present invention may inhibit DNA binding by cellular Stat3, synthetic Stat3, constitutively expressed Stat3, Stat monomers, Stat3 monomers, and/or Stat3 SH2 domain, in vivo, in vitro and/or in silico. In an aspect, a peptidomimetic of the present invention may inhibit transcription activities by cellular Stat3, synthetic Stat3, constitutively expressed Stat3, Stat monomers, Stat3 monomers, and/or Stat3 SH2 domain, in vivo, in vitro and/or in silico. In an aspect, a peptidomimetic of the present invention may inhibit the activities of cellular Stat3, synthetic Stat3, constitutively expressed Stat3, Stat monomers, Stat3 monomers, and/or Stat3 SH2 domain, in vivo, in vitro and/or in silico.

The present invention comprises methods and compositions comprising a Stat3 inhibitor. An example of a Stat3 inhibitor of the present invention is SPI, SEQ ID NO:1. An example of a Stat3 inhibitor of the present invention is SEQ ID NO:21. An example of a Stat3 inhibitor of the present invention is one or more of the disclosed polypeptide and sequences herein. In an aspect, a disclosed Stat3 inhibitor may inhibit phosphorylation of cellular Stat3, synthetic Stat3, constitutively expressed Stat3, Stat monomers, Stat3 monomers, and/or Stat3 SH2 domain, in vivo, in vitro and/or in silico. In an aspect, a Stat3 inhibitor of the present invention may inhibit DNA binding by cellular Stat3, synthetic Stat3, constitutively expressed Stat3, Stat monomers, Stat3 monomers, and/or Stat3 SH2 domain, in vivo, in vitro and/or in silico. In an aspect, a Stat3 inhibitor of the present invention may inhibit transcription activities by cellular Stat3, synthetic Stat3, constitutively expressed Stat3, Stat monomers, Stat3 monomers, and/or Stat3 SH2 domain, in vivo, in vitro and/or in silico. In an aspect, a Stat3 inhibitor of the present invention may inhibit the activities of cellular Stat3, synthetic Stat3, constitutively expressed Stat3, Stat monomers, Stat3 monomers, and/or Stat3 SH2 domain, in malignant cells, in cells that reproduce aberrantly, in cancerous cells, in normal cells, in transformed cells, in tumors, in individuals comprising cells that reproduce aberrantly, cancerous cells, and/or normal cells.

The present invention comprises methods and compositions comprising a polypeptide that inhibits Stat3 activation. An example of a polypeptide of the present invention is SPI, SEQ ID NO:1. An example of a polypeptide of the present invention is SEQ ID NO:21. An example of a polypeptide of the present invention is one or more of the disclosed polypeptides and sequences herein. In an aspect, a disclosed polypeptide may inhibit Stat3 activation by inhibiting cellular Stat3, synthetic Stat3, constitutively expressed Stat3, Stat monomers, Stat3 monomers, and/or Stat3 SH2 domain, in vivo, in vitro and/or in silico, or in a combination thereof.

The present invention comprises methods and compositions comprising a polypeptide that is cell membrane permeable. An example of a cell permeable polypeptide of the present invention is SPI, SEQ ID NO:1. An example of a cell permeable polypeptide of the present invention is SEQ ID NO:21. An example of a cell permeable polypeptide of the present invention is one or more of the disclosed polypeptides and sequences herein. A cell permeable polypeptide of the present invention is cell membrane permeable, wherein the cell membrane is the cell plasma membrane. A cell permeable polypeptide of the present invention is cell membrane permeable, wherein the cell membrane is a cytoplasmic membrane. A cell permeable polypeptide of the present invention is cell membrane permeable, wherein the cell membrane is a nuclear membrane.

The present invention comprises methods and compositions comprising a Stat3 SH2 domain mimicking polypeptide that comprises SEQ ID NO: 1. An example of a Stat3 SH2 domain mimicking polypeptide of the present invention is SPI, SEQ ID NO:1. An example of a Stat3 SH2 domain mimicking polypeptide of the present invention is SEQ ID NO:21. An example of a Stat3 SH2 domain mimicking polypeptide of the present invention is one or more of the disclosed polypeptide and sequences herein. In an aspect, a Stat3 SH2 domain mimicking polypeptide is a variant of SEQ ID NO:1. In an aspect, the polypeptide comprises some degree of homology, such as greater than 75% homology, to SEQ ID NO:1. In an aspect, the polypeptide comprises some degree of homology, such as greater than 75% homology, to SEQ ID NO:21.

Disclosed herein are polypeptides comprising SEQ ID NO:1. One of ordinary skill in the art at the time of the invention would have understood that other variations can occur in the sequence of SEQ ID NO:1. Some variations do not affect its functionality, while others affect the functionality. If the functionality is affected in a positive way, the variation is selected for. Specifically disclosed are peptide variants that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to SEQ ID NO:1. Disclosed are peptide variants that have 70-74, 75-79, 80-84, 85-89, 90-94, 95-99 percent homology to SEQ ID NO:1. Disclosed peptide variants comprise 70-99, 75-95, or 80-90 percent homology to SEQ ID NO:1, or 70-80, 80-90, or 90-100 percent homology to SEQ ID NO:1.

The present invention comprises Stat3 mimetic polypeptides that have fewer than 28 amino acids. Disclosed herein are polypeptides comprising SEQ ID NO:21. One of ordinary skill in the art at the time of the invention would have understood that other variations can occur in the sequence of SEQ ID NO:21. Some variations do not affect its functionality, while others affect the functionality. If the functionality is affected in a positive way, the variation is selected for. Specifically disclosed are peptide variants that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to SEQ ID NO:21. Disclosed are peptide variants that have 70-74, 75-79, 80-84, 85-89, 90-94, 95-99 percent homology to SEQ ID NO:21. Disclosed peptide variants comprise 70-99, 75-95, or 80-90 percent homology to SEQ ID NO:21, or 70-80, 80-90, or 90-100 percent homology to SEQ ID NO:21.

Those of skill in the art readily understand how to determine the homology between two or more proteins or two or more nucleic acids. For example, the homology can be calculated after aligning the two sequences so that the homology is at its highest level. Another way of calculating homology can be performed by published algorithms. Optimal alignment of sequences for comparison may be conducted by the local homology algorithm of Smith et al., 1981, by the homology alignment algorithm of Needleman et al., 1970, by the search for similarity method of Pearson et al., 1988, by computerized implementations of these algorithms (GAP, BESTFIT, FASTA, and TFASTA in the Wisconsin Genetics Software Package, Genetics Computer Group, 575 Science Dr., Madison, Wis.), or by inspection.

The present invention comprises methods and compositions comprising Stat3 SH2 domain mimicking polypeptide comprising modified amino acids. In an aspect, the polypeptide comprises at least one modified amino acid. For example, a polypeptide comprising the sequence of SEQ ID NO:1 can comprise one or more modified amino acids. A polypeptide of the present invention comprising the sequence of SEQ ID NO:21 can comprise one or more modified amino acids. In an aspect, a polypeptide is modified at the amino terminus or at the carboxy terminus. In an aspect, both the amino and carboxy termini are modified. In an aspect, a polypeptide comprises at least one modified amino acid that is not at the carboxy or amino termini.

The present invention comprises methods and compositions comprising a Stat3 SH2 domain mimicking polypeptide comprising at least one label. The disclosed compositions and polypeptides can, for example, be labeled so that the label or moiety that can be selectively detected, such as in an assay. Examples include without limitation, radiolabels, (e.g., ³H, ¹⁴C, ³⁵S, ¹²⁵I, ¹³¹I) affinity tags (e.g., biotin/avidin or streptavidin, binding sites for antibodies, metal binding domains, epitope tags, FLASH binding domains (U.S. Pat. Nos. 6,451,569, 6,054,271, 6,008,378, and 5,932,474 discussing glutathione or maltose binding domains), fluorescent or luminescent moieties (e.g., fluorescein and derivatives, GFP, rhodamine and derivatives, lanthanides etc.), and enzymatic moieties (e.g., horseradish peroxidase, beta-galactosidase, beta-lactamase, luciferase, alkaline phosphatase). Such detectable labels can be formed in situ, for example, through use of an unlabeled primary antibody which can be detected by a secondary antibody having an attached detectable label.

The present invention comprises methods and compositions comprising a Stat3 inhibitor comprising a Stat3 SH2 domain mimicking polypeptide. An example of a Stat3 inhibitor comprising a Stat3 SH2 domain mimicking polypeptide of the present invention is SPI, SEQ ID NO:1. An example of a Stat3 inhibitor comprising a Stat3 SH2 domain mimicking polypeptide of the present invention SEQ ID NO:1 or SEQ ID NO:21. An example of a Stat3 inhibitor comprising a Stat3 SH2 domain mimicking polypeptide of the present invention is one or more of the disclosed polypeptide and sequences herein.

The present invention comprises methods and compositions comprising an isolated nucleic acid encoding a Stat3 inhibitor. In an aspect, the isolated nucleic acid encodes a Stat3 SH2 domain mimicking polypeptide. In an aspect, the nucleic acid encodes a polypeptide with the amino acid sequence of SEQ ID NO:1 or SEQ ID NO:21. In an aspect, the nucleic acid encodes a polypeptide disclosed herein. Disclosed herein is an isolated nucleic acid encoding a Stat3 inhibitor that is cell membrane permeable. In an aspect, the cell membrane permeable polypeptide encoded by the isolated nucleic acid is distributed throughout a cell, and can be, for example, co-localized to the nucleus of a cell or co-localized at the plasma membrane.

The present invention comprises methods and compositions comprising vectors encoding a Stat3 inhibitor, for example, a Stat3 SH2 domain mimicking polypeptide, for example, SPI. Disclosed are expression vectors useful in eukaryotic host cells (yeast, fungi, insect, plant, animal, human or nucleated cells) which can also contain sequences necessary for the termination of transcription which may affect mRNA expression. Disclosed vectors comprise a nucleic acid encoding a Stat3 inhibitor. In an aspect, the nucleic acid of the vector encodes a Stat3 SH2 domain mimicking polypeptide. In an aspect, nucleic acid of the vector encodes a Stat3 inhibitor comprising a polypeptide comprising SEQ ID NO:1. In an aspect, the nucleic acid of the vector encodes a Stat3 SH2 domain mimicking polypeptide comprising at least SEQ ID NO:1. In an aspect, the vector comprises a nucleic acid encoding a Stat3 inhibitor that is at least 75 percent homologous to SEQ ID NO:1. In an aspect, the vector comprises a nucleic acid encoding a Stat3 SH2 domain mimicking polypeptide that is at least 75 percent homologous to SEQ ID NO:1. In an aspect, the homology of the disclosed Stat3 inhibitor or the disclosed Stat3 SH2 domain mimicking polypeptide is at least 70-80, or 80-90, or 90-100 percent homologous to SEQ ID NO: 1. In an aspect, the homology of the disclosed Stat3 inhibitor or the disclosed Stat3 SH2 domain mimicking polypeptide is at least 75, 80, 85, 90, 95, 96, 97, 98, or 99 percent homologous to SEQ ID NO:1.

The present invention comprises methods and compositions comprising vectors encoding a Stat3 inhibitor, for example, a portion of a Stat3 SH2 domain mimicking polypeptide. Disclosed are expression vectors useful in eukaryotic host cells (yeast, fungi, insect, plant, animal, human or nucleated cells) can also contain sequences necessary for the termination of transcription which may affect mRNA expression. The disclosed vector comprises a nucleic acid encoding a Stat3 inhibitor. In an aspect, the nucleic acid of the vector encodes a Stat3 SH2 domain mimicking polypeptide. In an aspect, nucleic acid of the vector encodes a Stat3 inhibitor comprising a polypeptide comprising SEQ ID NO:21. In an aspect, the nucleic acid of the vector encodes a Stat3 SH2 domain mimicking polypeptide comprising at least SEQ ID NO:21. In an aspect, the vector comprises a nucleic acid encoding a Stat3 inhibitor that is at least 75 percent homologous to SEQ ID NO:21. In an aspect, the vector comprises a nucleic acid encoding a Stat3 SH2 domain mimicking polypeptide that is at least 75 percent homologous to SEQ ID NO:21. In an aspect, the homology of the disclosed Stat3 inhibitor or the disclosed Stat3 SH2 domain mimicking polypeptide is at least 70-80, or 80-90, or 90-100 percent homologous to SEQ ID NO:21. In an aspect, the homology of the disclosed Stat3 inhibitor or the disclosed Stat3 SH2 domain mimicking polypeptide is at least 75, 80, 85, 90, 95, 96, 97, 98, or 99 percent homologous to SEQ ID NO:21.

The present invention comprises methods and compositions comprising an isolated nucleic acid encoding any one or more of the polypeptides disclosed herein. In an aspect, the nucleic acid comprises DNA, RNA, and/or cDNA. It would be routine for one with ordinary skill in the art to make a nucleic acid that encodes the polypeptides disclosed herein since codons for each of the amino acids that make up the polypeptides are known. As non-limiting examples, the nucleic acids of the invention can be produced by recombinant, in vitro methods, or by chemical synthetic means using machines and standard chemistry which would be known to one of skill in the art, or by in vivo cellular synthesis. Methods of synthesizing nucleic acids would be well known to one of skill in the art, e.g., U.S. Pat. No. 6,472,184 and U.S. Pat. No. 6,444,111. These references are hereby incorporated by reference in their entireties.

Additionally, the invention provides a vector comprising the nucleic acid encoding any one or more of the polypeptides and peptides described herein. In an aspect, the invention provides a vector comprising a nucleic acid encoding at least one of the polypeptides of the present invention, e.g., SEQ ID NO:1. In an aspect, the invention provides a vector comprising a nucleic acid encoding a variant polypeptides of the present invention, e.g., a variant of SEQ ID NO:1. The vector can be a viral vector, a plasmid vector, a cosmid vector, an adenoviral vector, a phage vector, a retroviral vector, an adeno-associated viral (AAV) vector, or any other vector capable of including a nucleic acid encoding a peptide or polypeptide of the invention. The vector can be an expression vector that is intended and capable of integrating into a cell genome. Other useful virus vectors include retroviruses such as Moloney murine leukemia virus (MoMuLV); papovaviruses such as JC, SV40, polyoma, adenoviruses; Epstein-Barr Virus (EBV); papilloma viruses, e.g., bovine papilloma virus type I (BPV); vaccinia and poliovirus and other human and animal viruses.

Preferred promoters controlling transcription from vectors in mammalian host cells may be obtained from various sources, for example, the genomes of viruses such as: polyoma, Simian Virus 40 (SV40), adenovirus, retroviruses, hepatitis B virus and cytomegalovirus, or from heterologous mammalian promoters, e.g., beta actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII E or Sau3A restriction fragment. Promoters from the host cell or related species are useful herein.

Whether heterologous or homologous, enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene.

Whether homologous or heterologous, a promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

The disclosed vectors can comprise elements (such as, for example, promoters, enhancers, 3′-UTRs, LTRS, etc.) that are heterologous or homologous to the nucleic acid encoding a disclosed polypeptide of the present invention. The skilled person is familiar with the compositions and methods used to construct vectors comprising heterologous and homologous elements, such as, for example, a promoter, or enhancer, or 3′UTR, or LTR, or etc. that is homologous or heterologous to the sequence encoding the nucleic acid of interest.

The vectors used in host cells contain all or a part of a viral genome, such as long term repeats (“LTRs”), promoters (e.g., CMV promoters, SV40 promoter, RSV promoter), enhancers, and so forth. A non-limiting example of such adenoviruses which can be employed in the present invention are well-known in the art and include more than 40 different human adenoviruses, e.g., Ad12 (subgenus A), Ad3 and Ad7 (Subgenus B), Ad2 and Ad5 (Subgenus C), Ad8 (Subgenus D), Ad4 (Subgenus E), Ad40 (Subgenus F). When the host cell is a prokaryote, bacterial viruses, or phages, can be used to deliver the nucleic acid of the invention to the host cell. A non-limiting example of such vectors are vectors based upon, for example, lambda phage. In any case, the vector may comprise elements of more than one virus. The vector may additionally comprise a gene encoding a marker or reporter molecule to more easily trace expression of the vector.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

The present invention comprises host cells comprising one or more Stat3 SH2 domain mimicking polypeptides. In an aspect, host cells comprise an isolated nucleic acid encoding a Stat3 inhibitor. In an aspect, host cells comprise an isolated nucleic acid encoding a Stat3 SH2 domain mimicking polypeptide. In an aspect, host cells comprise a nucleic acid encoding a polypeptide with the amino acid sequence of SEQ ID NO:1, and in an aspect, the nucleic acid encodes a polypeptide comprising SEQ ID NO:1. In an aspect, host cells comprise a nucleic acid encoding a polypeptide with the amino acid sequence of SEQ ID NO:21, and in an aspect, the nucleic acid encodes a polypeptide comprising SEQ ID NO:21. Disclosed are host cells comprising a nucleic acid encoding a recombinant polypeptide. In an aspect, the recombinant polypeptide is a Stat3 SH2 domain mimicking polypeptide. Disclosed are host cells comprising Stat3 inhibiting polypeptides.

In an aspect, host cells of the present invention may comprise an isolated nucleic acid encoding a Stat3 inhibitor that is cell membrane permeable. In an aspect, host cells comprise an isolated nucleic acid encoding a Stat3 SH2 domain mimicking polypeptide. In an aspect, the cell membrane is a plasma membrane. In an aspect, the cell membrane is a cytoplasmic membrane. In an aspect, the cell membrane is a nuclear membrane.

The present invention comprises methods and compositions comprising host cells comprising polypeptides. In an aspect, host cells comprise a polypeptide that inhibits Stat3. In an aspect, host cells comprise an isolated nucleic acid encoding a Stat3 SH2 domain mimicking polypeptide. In an aspect, host cells comprise a nucleic acid encoding a polypeptide with the amino acid sequence of SEQ ID NO:1, and in an aspect, the nucleic acid encodes a polypeptide comprising SEQ ID NO:1. In an aspect, host cells comprise a nucleic acid encoding a polypeptide with the amino acid sequence of SEQ ID NO:21 and in an aspect, the nucleic acid encodes a polypeptide comprising SEQ ID NO:21. Disclosed are host cells comprising a nucleic acid encoding a recombinant polypeptide. In an aspect, the recombinant polypeptide is a Stat3 SH2 domain mimicking polypeptide.

In an aspect, host cells comprise an isolated nucleic acid encoding a Stat3 inhibitor that is cell membrane permeable. In an aspect, host cells comprise an isolated nucleic acid encoding a Stat3 SH2 domain mimicking polypeptide. In an aspect, the cell membrane is a plasma membrane. In an aspect, the cell membrane is a cytoplasmic membrane. In an aspect, the cell membrane is a nuclear membrane.

The present invention comprises methods and compositions comprising a composition comprising a Stat3 SH2 domain mimicking polypeptide that inhibits Stat3 activation. In an aspect, the composition further comprises a pharmaceutically acceptable carrier. The inhibition of Stat3 activation can occur in vivo, in vitro, or in silico, or in a combination thereof. The present invention comprises methods and compositions comprising a composition comprising a Stat3 inhibitor, wherein the inhibitor comprises SEQ ID NO:1 The present invention comprises methods and compositions comprising a composition comprising a Stat3 inhibitor, wherein the inhibitor comprises SEQ ID NO:21 A disclosed Stat3 inhibitor can be cell permeable, and can permeate a plasma membrane, a cytoplasmic membrane, the nuclear membrane, or one or all membranes.

The present invention comprises methods and compositions comprising a composition that inhibits Stat3. In an aspect, the composition of Stat3 inhibitor comprises a polypeptide. In an aspect, the composition further comprises a pharmaceutically acceptable carrier. The Stat3 inhibitors disclosed herein can be recombinant or synthetic polypeptides. The polypeptide is SPI, or can comprise SEQ ID NO:1, or portions thereof. In an aspect, a disclosed Stat3 inhibitor is FISKERERAILSTKPPGTFLLRFSESSK. In an aspect, a polypeptide can comprise SEQ ID NO:21. In an aspect, a disclosed Stat3 inhibitor is ISKERERAILSTKPP.

Compositions disclosed herein, including but not limited to, Stat3 inhibitors, or antibodies that specifically bind to Stat3 inhibitors disclosed herein, can be used therapeutically in combination with a pharmaceutically acceptable carrier. Suitable carriers and their formulations are described in Remington 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid and phosphoric acid, and/organic acids such as formic acid, acetic acid, propionic acid, glycolic acid, lactic acid, pyruvic acid, oxalic acid, malonic acid, succinic acid, maleic acid and fumaric acid, or by reaction with an inorganic base such as sodium hydroxide, ammonium hydroxide, and potassium hydroxide, and/organic bases such as mono-, di-, trialkyl and aryl amines and substituted ethanolamines.

The present invention comprises methods and compositions comprising a monoclonal antibody that specifically binds to a Stat3 SH2 domain mimicking polypeptide or an antigenic portion thereof. Compositions comprise antibodies, whether polyclonal or monoclonal, or fragments or subunits of antibodies, that specifically bind to SPI, to polypeptides comprising SEQ ID NO:1, or other polypeptides and/or sequences disclosed herein, such polypeptides comprising SEQ ID NO:21. In an aspect, an antibody binds to a recombinant SH2 domain mimicking polypeptide. In an aspect, an antibody binds to a synthetic SH2 domain mimicking polypeptide. The present invention comprises a monoclonal or polyclonal antibody that specifically binds to a Stat3 inhibitor. In an aspect, an antibody binds to a Stat3 inhibitor that is a polypeptide. A Stat3 inhibitor disclosed herein can be recombinant or synthetic polypeptide. In an aspect, the antibody can bind to a polypeptide comprising SEQ ID NO:1, or portions thereof. In an aspect, antibody can bind to a polypeptide of SEQ ID NO:21, or portions thereof. In an aspect, an antibody binds to the disclosed Stat3 inhibitor wherein the Stat3 inhibitor is FISKERERAILSTKPPGTFLLRFSESSK or ISKERERAILSTKPP.

The present invention comprises methods and compositions for diagnosing cancer comprising using a monoclonal antibody to determine the levels of Stat3 in a sample, subject, or patient. In an aspect, the monoclonal antibody binds to a polypeptide of SEQ ID NO:1. In an aspect, the monoclonal antibody binds to a polypeptide comprising SEQ ID NO:1. In an aspect, the monoclonal antibody can bind to a polypeptide comprising SEQ ID NO:21. The present invention comprises methods and compositions for diagnosing cancer comprising using polyclonal antibodies to determine the levels of Stat3 in a sample, subject, or patient. In an aspect, the polyclonal antibodies binds to a polypeptide of SEQ ID NO:1. In an aspect, the polyclonal antibodies bind to a polypeptide comprising SEQ ID NO:1.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a Stat3 SH2 domain mimicking polypeptide disclosed herein. For example, in an aspect, an antibody binds to or interacts with the polypeptide represented by SEQ ID NO:1. The present invention comprises antibodies that bind to or interact with a portion of SEQ ID NO:1 In an aspect, an antibody binds to or interacts to a Stat3 inhibitor comprising SEQ ID NO:1.

In an aspect, an antibody binds to or interacts with a polypeptide that has a certain homology to the polypeptide represented by SEQ ID NO:1, such as a polypeptide that has 70, 75, 80, 85, 90, or 95 percent homology, or a percent in between 70-99 percent homology, to the polypeptide of SEQ ID NO:1. Specifically disclosed are antibodies that bind to or interact with peptide variants that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to SEQ ID NO:1. Disclosed are antibodies that bind to or interact with peptide variants that have 70-74, 75-79, 80-84, 85-89, 90-94, 95-99 percent homology to SEQ ID NO:1. Disclosed herein are antibodies that bind to or interact with peptide variants that have 70-99, 75-95, or 80-90 percent homology to SEQ ID NO:1, or 70-80, 80-90, or 90-100 percent homology to SEQ ID NO:1.

An aspect of the present invention comprises an antibody that binds to or interacts with a polypeptide comprising SEQ ID NO:1 and additional amino acids on the amino end of SEQ ID NO:1, or on the carboxy end of SEQ ID NO:1, or on both the amino end and the carboxy end of SEQ ID NO:1. For example, disclosed herein is an antibody that binds to or interacts with a polypeptide comprising, for example, 20, 19, 18, 17, 16, 15, 14, 13, 12, 11, 10, 9, 8, 7, 6, 5, 4, 3, 2, or 1 additional amino acids that are on the amino end to SEQ ID NO:1. Further disclosed is an antibody that binds to or interacts with a variant polypeptide comprising, for example, 20, 19, 18, 17, 16, 15, 14, 13, 12, 11, 10, 9, 8, 7, 6, 5, 4, 3, 2, or 1 additional amino acids that are on the carboxy end of SEQ ID NO:1. Disclosed herein is an antibody that binds to or interacts with a polypeptide comprising a combination of 20, 19, 18, 17, 16, 15, 14, 13, 12, 11, 10, 9, 8, 7, 6, 5, 4, 3, 2, or 1 additional amino acids, or a combination thereof, that are on the amino and carboxy ends of SEQ ID NO:1.

An aspect of the present invention comprises an antibody that binds to or interacts with a polypeptide comprising SEQ ID NO:1 and additional amino acids on the amino end of SEQ ID NO:1, or on the carboxy end of SEQ ID NO:1, or on both the amino end and the carboxy end of SEQ ID NO:1. For example, disclosed herein is an antibody that binds to or interacts with a polypeptide comprising, for example, 15-20, or 10-15, or 5-10, or 1-5 additional amino acids that are on the amino end of SEQ ID NO:1. Further disclosed is an antibody that binds to or interacts with a polypeptide comprising, for example, 15-20, or 10-15 or 5-10, or 1-5 additional amino acids that are on the carboxy end of SEQ ID NO:1. Disclosed herein is an antibody that binds to or interacts with a polypeptide comprising SEQ ID NO:1 and a combination of 15-20, or 10-15, or 5-10, or 1-5 additional amino acids, or a combination thereof, that are both on the amino end and the carboxy end of SEQ ID NO:1.

The present invention comprises methods and compositions comprising antibodies that bind to or interact with SEQ ID NO:21. The present invention comprises an antibody that binds to or interacts with a portion of SEQ ID NO: 21. In an aspect, an antibody binds to or interacts to a Stat3 inhibitor comprising SEQ ID NO:21. An aspect of the present invention comprises an antibody that binds to or interacts with a polypeptide comprising SEQ ID NO:21. In an aspect, an antibody binds to or interacts with a polypeptide that has a certain homology to the polypeptide represented by SEQ ID NO:21, such as a polypeptide that has 70, 75, 80, 85, 90, or 95 percent homology, or a percent in between 70-99 percent homology, to the polypeptide of SEQ ID NO:21. Specifically disclosed are antibodies that bind to or interact with peptide variants that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to SEQ ID NO:21. Disclosed are antibodies that bind to or interact with peptide variants that have 70-74, 75-79, 80-84, 85-89, 90-94, 95-99 percent homology to SEQ ID NO:21. Disclosed herein are antibodies that bind to or interact with peptide variants that have 70-99, 75-95, or 80-90 percent homology to SEQ ID NO:21, or 70-80, 80-90, or 90-100 percent homology to SEQ ID NO:21.

The antibodies of the present invention can bind to or interact with one or more epitopes of a disclosed polypeptide. In an aspect, the antibodies bind to or interact with specific epitopes of a polypeptide comprising SEQ ID NO:1. For example, the antibodies bind to or interact with one or more specific amino acid residues of SEQ ID NO:1. In an aspect, the residues may comprise a Lysine residue at position 591, or residues may comprise an Arginine at position 609, or residues may comprise a Serine at position 611, or residues may comprise a Serine at position 613, or a combination thereof. The antibodies of the present invention can recognize one or more epitopes of a disclosed polypeptide, for example, SPI.

Antibodies of the present invention can bind to or interact with one or more epitopes of a disclosed polypeptide. In an aspect, antibodies bind to or interact with specific epitopes of a polypeptide comprising SEQ ID NO:21. For example, antibodies bind to or interact with a specific amino acid residue of SEQ ID NO:21. Antibodies of the present invention can recognize one or more epitopes of a disclosed polypeptide.

Disclosed antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (U.S. Pat. No. 4,816,567 and Morrison et al., 1984).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. One of ordinary skill in the art knows how to make or produce monoclonal antibodies, which specifically bind to a polypeptide having a known amino acid sequence. (e.g., Steplewski et al., 1985; Spira et al., 1984; WO 86/01533 (1986); U.S. Pat. No. 6,458,592). The monoclonal antibody, in some aspects, can be chimeric (e.g., U.S. Pat. No. 5,843,708), humanized (e.g., U.S. Pat. No. 6,423,511), primatized (e.g., U.S. Pat. No. 6,113,898), and/or linked to other polypeptides as fusion proteins. Portions of the monoclonal antibody can also be useful, either alone or linked to other proteins. These portions include, but are not limited to Fab (Fab′)₂, Fv, etc. In an aspect, the monoclonal antibody can be linked to a carrier (e.g., water, buffered water, 0.4% saline, 0.3% glycine, and the like) or can be associated with an adjuvant (e.g., biliverdin, bilirubin, biotin, carnosine, chitin, etc.). Adjuvants have been used experimentally to promote a generalized increase in immunity against unknown antigens (e.g., U.S. Pat. No. 4,877,611).

Methods for humanizing non-human antibodies are well known in the art. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response. Humanized antibodies can be generated according to the methods of Winter and co-workers (Jones et al., 1986; Riechmann et al., 1988; Verhoeyen et al., 1988) by substituting rodent CDRs or CDR sequences for the corresponding sequences of a human antibody. Methods that can be used to produce humanized antibodies are also described in U.S. Pat. Nos. 4,816,567, 5,565,332, 5,721,367, 5,837,243, 5,939,598, 6,130,364, and 6,180,377. These methods can be used to generate, for example, a humanized antibody that binds to or interacts with the polypeptide represented by SEQ ID NO:1 or a portion thereof, or that binds to or interacts with a Stat3 inhibitor comprising SEQ ID NO:1. In an aspect, a humanized antibody binds to or interacts with a polypeptide that has a certain homology to the polypeptide represented by SEQ ID NO:1, such as a polypeptide that has 70, 75, 80, 85, 90, or 95 percent homology, or a percent in between 70-99 percent homology, to the polypeptide of SEQ ID NO: 1. In an aspect, a humanized antibody binds to or interacts with a Stat3 inhibitor comprising SEQ ID NO:1, or with a Stat3 inhibitor comprising a polypeptide that has 70, 75, 80, 85, 90, or 95 percent homology, or a percent in between 70-99 percent homology, to the polypeptide of SEQ ID NO:1.

Administration of the antibodies disclosed herein is also known to the art. Nucleic acid approaches for antibody delivery also exist. The anti-Stat3 SH2 domain mimicking polypeptide antibodies and antibody fragments disclosed herein can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The anti-Stat3 SH2 domain mimicking polypeptide antibodies and antibody fragments disclosed herein can be administered to patients or subjects as therapeutic compositions.

The present invention comprises methods and compositions for modulating Stat production in a subject with an aberrant level of Stat protein, such as an individual with cancer. The present invention comprises methods of modulating aberrantly produced Stat3 in at least one cell of an individual, comprising, administering, to the individual an effective amount of a composition comprising SPI, or polypeptides comprising SEQ ID NO:1, or other polypeptides and/or sequences disclosed herein, such as SEQ ID NO:21, or combinations thereof. Methods may further comprise administering chemotherapeutic agents in conjunction, at the same time, following or sequentially, with SH2 domain mimicking polypeptide compositions disclosed herein. Such methods may comprise aberrant levels of Stat proteins, such as Stat3, in an individual with cancer. Cancer may comprise uncontrolled cellular proliferation, for example, cancer, wherein the cancer is head and neck, breast, prostate, renal cell, melanoma, ovarian, lung, leukemia, lymphoma and multiple myeloma, pancreatic, and non-small cell lung cancer. Methods of the present invention comprise treatment of cancer that is chemotherapy-resistant cancer.

The present invention comprises methods and compositions for modulating Stat production in a subject with an aberrant level of Stat protein, such as a subject with cancer. The present invention comprises methods of treating cancer in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in the cancer, or other changes. Methods may further comprise administering chemotherapeutic agents in conjunction, at the same time, following or sequentially, with treatment of cancer with SH2 domain mimicking polypeptide compositions disclosed herein. Cancer may comprise uncontrolled cellular proliferation, for example, cancer, wherein the cancer is head and neck, breast, prostate, renal cell, melanoma, ovarian, lung, leukemia, lymphoma and multiple myeloma, pancreatic, and non-small cell lung cancer. Methods of the present invention comprise treatment of cancer that is chemotherapy-resistant cancer.

The present invention comprises methods and compositions for diagnosing cancer in a subject by determining the presence or amount of an aberrant level of Stat protein. The present invention comprises methods of detecting aberrantly produced Stat3 in at least one cell of a subject. The method may comprise detectably labeled SPI, or polypeptides comprising SEQ ID NO:1, or other polypeptides and/or sequences disclosed herein, such as SEQ ID NO:21, or combinations thereof. The method may comprise use of an antibody or fragment thereof to SPI, or polypeptides comprising SEQ ID NO:1, or other polypeptides and/or sequences disclosed herein, such as SEQ ID NO:21, or combinations thereof.

The present invention comprises methods and compositions for prognosis of cancer in a subject by determining the presence or amount of an aberrant level of Stat protein. The present invention comprises methods of detecting aberrantly produced Stat3 in at least one cell of a subject. The method may comprise detectably labeled SPI, or polypeptides comprising SEQ ID NO:1, or other polypeptides and/or sequences disclosed herein, such as SEQ ID NO:21, or combinations thereof. The method may comprise use of an antibody or fragment thereof to SPI, or polypeptides comprising SEQ ID NO:1, or other polypeptides and/or sequences disclosed herein, or combinations thereof.

The present invention comprises methods and compositions for determining effectiveness of cancer treatment in a subject by determining the presence of, amount of, or change in, an aberrant level of Stat protein, during or after a course of treatment of the cancer, such as with an anti-cancer therapeutic and/or treatment with Stat3 SH2 domain mimicking polypeptide comprising SPI, or polypeptides comprising SEQ ID NO:1, or other polypeptides and/or sequences disclosed herein, such as SEQ ID NO:21, or combinations thereof. The present invention comprises methods of detecting the level or amount of, or activity of, Stat3 in at least one cell of a subject. The method may comprise detectably labeled SPI, or polypeptides comprising SEQ ID NO:1, or other polypeptides and/or sequences disclosed herein, or combinations thereof. The method may comprise use of an antibody or fragment thereof to SPI, or polypeptides comprising SEQ ID NO:1, or other polypeptides and/or sequences disclosed herein, such as SEQ ID NO:21, or combinations thereof.

Disclosed compositions can be used to treat a patient or subject. Treatment as used herein can refer to various types of compositions, techniques, therapies, and devices that can be used to affect aberrant cell growth, tumor development, and cancer. For example, treatment can comprise a chemical, a pharmaceutical agent, or combinations thereof, which can be administered to a subject to treat aberrant cell growth, tumor development, and cancer. Treatment can comprise surgical intervention. Treatment can comprise therapy. Treatments can be delivered or exercised alone or can be delivered or exercised in combination with one or more other forms of treatment. Treatment can be repeatedly or continuously delivered. Such treatment can affect the subject's susceptibility for aberrant cell growth, tumor development, and cancer, or to partially or fully reverse the effects of aberrant cell growth, tumor development, and cancer.

Provided herein is a method of treating tumor formation and development or cancer in a subject or a patient, comprising administering to patient or subject the disclosed compositions or polypeptides. The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The term “subject” means any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. Subject includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject may to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The peptides, polypeptides, nucleic acids, antibodies, vectors and therapeutic compositions of the invention can be combined with other well-known anti-tumor or anti-cancer therapies already in use. The disclosed compositions, polypeptides, and nucleic acids of the invention can generate an additive or a synergistic effect with current treatments. For example, the following are lists of anti-cancer (anti-neoplastic) drugs that can be used in conjunction with the presently disclosed polypeptides and compositions: Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 1131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The compositions provided herein can further comprise one or more additional radiosensitizers. Examples of known radiosensitizers include gemcitabine, 5-fluorouracil, pentoxifylline, and vinorelbine.

The majority of chemotherapeutic drugs can be divided in to: alkylating agents, antimetabolites, anthracyclines, plant alkaloids, topoisomerase inhibitors, monoclonal antibodies, and other antitumor agents. All of these drugs affect cell division or DNA synthesis. Some newer agents do not directly interfere with DNA. These include the new tyrosine kinase inhibitor imatinib mesylate, which directly targets a molecular abnormality in certain types of cancer (chronic myelogenous leukemia, gastrointestinal stromal tumors). In addition, some drugs can be used which modulate tumor cell behaviour without directly attacking those cells. Hormone treatments fall into this category of adjuvant therapies.

The chemotherapeutic of the disclosed method can be an alkylating agent. Alkylating agents are so named because of their ability to add alkyl groups to many electronegative groups under conditions present in cells. Cisplatin and carboplatin, as well as oxaliplatin are alkylating agents. Other agents are mechloethamine, cyclophosphamide, chlorambucil. They work by chemically modifying a cell's DNA.

The chemotherapeutic of the disclosed method can be an anti-metabolite. Anti-metabolites masquerade as purine (azathioprine, mercaptopurine) or pyrimidine—which become the building blocks of DNA. They prevent these substances becoming incorporated in to DNA during the ‘S’ phase (of the cell cycle), stopping normal development and division. They also affect RNA synthesis. Due to their efficiency, these drugs are the most widely used cytostatics.

The chemotherapeutic of the disclosed method can be a plant alkaloids or terpenoids. These alkaloids are derived from plants and block cell division by preventing microtubule function. Microtubules are vital for cell division and without them it can not occur. The main examples are vinca alkaloids and taxanes.

The chemotherapeutic of the disclosed method can be a vinca alkaloid. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine, Vinblastine, Vinorelbine, Vindesine, and Podophyllotoxin. Podophyllotoxin is a plant-derived compound used to produce two other cytostatic drugs, etoposide and teniposide. They prevent the cell from entering the G1 phase (the start of DNA replication) and the replication of DNA (the S phase). The exact mechanism of its action still has to be elucidated. The substance has been primarily obtained from the American Mayapple (Podophyllum peltatum). A rare Himalayan Mayapple (Podophyllum hexandrum) contains it in a much greater quantity, but as the plant is endangered, its supply is limited. Studies have been conducted to isolate the genes involved in the substance's production, so that it could be obtained recombinantly.

The chemotherapeutic of the disclosed method can be a taxane. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

The chemotherapeutic of the disclosed method can be a topoisomerase inhibitor. Topoisomerases are essential enzymes that maintain the topology of DNA Inhibition of type I or type II topoisomerases interferes with both transcription and replication of DNA by upsetting proper DNA supercoiling. Some type I topoisomerase inhibitors include the camptothecins irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

The chemotherapeutic of the disclosed method can be an antitumor antibiotic (Antineoplastics).

The chemotherapeutic of the disclosed method can be an (monoclonal) antibody. Monoclonal antibodies work by targeting tumor specific antigens, thus enhancing the host's immune response to tumor cells to which the agent attaches itself Examples are trastuzumab (Herceptin), cetuximab, and rituximab (Rituxan or Mabthera). Bevacizumab is a monoclonal antibody that does not directly attack tumor cells but instead blocks the formation of new tumor vessels.

The chemotherapeutic of the disclosed method can be a hormonal therapy. Several malignancies respond to hormonal therapy. Strictly speaking, this is not chemotherapy. Cancer arising from certain tissues, including the mammary and prostate glands, may be inhibited or stimulated by appropriate changes in hormone balance. Steroids (often dexamethasone) can inhibit tumor growth or the associated edema (tissue swelling), and may cause regression of lymph node malignancies. Prostate cancer is often sensitive to finasteride, an agent that blocks the peripheral conversion of testosterone to dihydrotestosterone. Breast cancer cells often highly express the estrogen and/or progesterone receptor. Inhibiting the production (with aromatase inhibitors) or action (with tamoxifen) of these hormones can often be used as an adjunct to therapy. Gonadotropin-releasing hormone agonists (GnRH), such as goserelin possess a paradoxic negative feedback effect followed by inhibition of the release of FSH (follicle-stimulating hormone) and LH (luteinizing hormone), when given continuously. Some other tumors are also hormone dependent, although the specific mechanism is still unclear.

In general, when used for treatment, the therapeutic compositions may be administered orally, parenterally (e.g., intravenously or subcutaneous administration), by intramuscular injection, by intraperitoneal injection, transdermally, extracorporeally, by intracavity administration, transdermally, or topically or the like, including topical intranasal administration or administration by inhalant. The topical administration can be ophthalmically, vaginally, rectally, or intranasally. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. Parenteral administration includes use of a slow release, a time release or a sustained release system such that a constant dosage is maintained.

The term “therapeutically effective” means that the amount of the composition used is of sufficient quantity to ameliorate one or more causes or symptoms of a disease or disorder, such as aberrant cell growth, tumor development, and cancer. Such amelioration only requires a reduction or alteration, not necessarily elimination. Effective dosages and schedules for administering the disclosed compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

The specific effective amount of a composition comprising the disclosed polypeptides or nucleic acids for any particular subject or patient will depend upon a variety of factors including the disease or disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts.

For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular subject or patient: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

The effective amount of the disclosed composition may be given daily, every other day, weekly, monthly, bi-monthly, every other monthly, yearly, or at any other interval that is determined by the physician or provider to be effective. For example, the effective daily dose can be divided into multiple doses for purposes of administration. Consequently, single dose compositions can contain such amounts or submultiples thereof to make up the daily dose. Disclosed compositions can also be administered as part of a combination of anti-tumor or anti-cancer treatments. In an aspect, disclosed compositions can be administered to the subject or patient prior to treatment with an anti-tumor or anti-cancer treatment. In an aspect, disclosed compositions can be administered concurrently with the anti-tumor or anti-cancer treatment. In an aspect, disclosed composition can be administered subsequent to the anti-tumor or anti-cancer treatment. In an aspect, the patient or subject receives both treatments on an alternating or rotating schedule. In an aspect, the subject or patient receives a singular treatment with the disclosed composition. In an aspect, the subject or patient receives at least one treatment with the disclosed composition. In an aspect, the subject or patient receives at least one treatment with the disclosed composition and at least one other anti-tumor or anti-cancer treatment.

In a further aspect, an effective amount can be determined by preparing a series of compositions comprising varying amounts of the disclosed compositions such as the disclosed polypeptides and nucleic acids and determining the release characteristics in vivo and in vitro and matching these characteristics with specific pharmaceutical delivery needs, inter alia, subject body weight, disease condition and the like.

The dosage can be adjusted by the individual physician or the subject in the event of any counter-indications. Dosage can vary, and can be administered in one or more dose administrations daily, for one or several days. Guidance can be found in the literature for appropriate dosages for given classes of pharmaceutical products.

The present invention comprises methods and compositions for assays for determining Stat protein levels, or for identifying Stat protein inhibitors. The present invention comprises methods of screening for an inhibitor of Stat3, comprising, (a) providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof (b) providing a test compound, and (c) assaying binding of the test compound to the Stat3 SH2 domain mimicking polypeptide, wherein if the test compound binds to the Stat3 SH2 domain mimicking polypeptide, the test compound is an inhibitor of Stat3.

The present invention comprises methods and compositions for determining Stat protein levels in a cell, in an in vitro or in silico assay, in a subject, in a sample from a subject, or from other sources. A method comprises determining in a sample the Stat protein levels, for example, the Stat3 protein levels, by use of an antibody to a Stat3 SH2 domain mimicking polypeptide comprising SPI, or polypeptides comprising SEQ ID NO:1, or other polypeptides and/or sequences disclosed herein, such as SEQ ID NO:21, or combinations thereof. Methods for protein identification or determination of levels of a protein by use of an antibody are known to those skilled in the art. For example, a competitive immunoassay wherein a Stat3 SH2 domain mimicking polypeptide comprising SPI, or polypeptides comprising SEQ ID NO:1, or other polypeptides and/or sequences disclosed herein, such as SEQ ID NO:21, or combinations thereof, serve as known protein amounts are known to those skilled in the art.

A polypeptide of the present invention can be used in a competitive assay. For example, a polypeptide of the present invention can be used for measuring the amount of Stat3 in a sample.

The antibodies of the present invention can be employed in any known assay method, such as competitive binding assays, direct and indirect sandwich assays, and immunoprecipitation assays for the detection and quantitation of Stat3 in vitro and in vivo. The antibodies will bind Stat3 with an affinity that is appropriate for the assay method being employed.

For diagnostic applications, antibodies of the present invention may be labeled with a detectable moiety. The detectable moiety can be any one that is capable of producing, either directly or indirectly, a detectable signal. For example, the detectable moiety may be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, beta-galactosidase, or horseradish.

Competitive binding assays rely on the ability of a labeled standard (e.g., a Stat3 SH2 domain mimicking polypeptide, or an immunologically reactive portion thereof) to compete with the test sample analyte for binding with a limited amount of antibodies of the present invention. The amount of Stat3 in the test sample is inversely proportional to the amount of standard that becomes bound to the antibodies. To facilitate determining the amount of standard that becomes bound, the antibodies may be insolubilized before or after the competition, so that the standard and analyte that are bound to the antibodies may be separated from the standard and analyte which remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents, including antibodies of the present invention, are also useful for in vivo imaging. An antibody labeled with a detectable moiety may be administered to an animal or subject, preferably into the bloodstream, and the presence and location of the labeled antibody in the host assayed. The antibody may be labeled with any moiety that is detectable in an animal, whether by nuclear magnetic resonance, radiology, or other detection means known in the art.

Selective binding agents of the invention, including antibodies, may be used as therapeutics. These therapeutic agents are generally agonists or antagonists, in that they either enhance or reduce, respectively, at least one of the biological activities of Stat3. In an aspect, antagonist antibodies of the invention are antibodies or binding fragments thereof which are capable of specifically binding to Stat3 and which are capable of inhibiting or eliminating the functional activity of Stat3 in vivo or in vitro. In an aspect, the selective binding agent, e.g., an antagonist antibody, will inhibit the functional activity of Stat3 by at least about 50%. In an aspect, the selective binding agent may be a polypeptide of the present invention (such as a polypeptide comprising a Stat3 SH2 domain mimicking polypeptide, or SPI, or a polypeptide comprising SEQ ID NO:1, or a polypeptide comprising SEQ ID NO:21) that is capable of interacting with Stat3 binding partner (a ligand or receptor) thereby inhibiting or eliminating Stat3 activity in vitro or in vivo. Selective binding agents, including agonist and antagonist anti-Stat3 SH2 domain mimicking polypeptide antibodies, are identified by screening assays that are well known in the art.

The present invention comprises methods and compositions for determining cellular activities and pathways in which Stat proteins function comprising using detectable polypeptide, such as a labeled polypeptide, comprising a Stat3 SH2 domain mimicking polypeptide comprising SPI, or polypeptides comprising SEQ ID NO:1, or other polypeptides and/or sequences disclosed herein, or combinations thereof A method comprises providing a detectable Stat3 SH2 domain mimicking polypeptide comprising SPI, or polypeptides comprising SEQ ID NO:1, or other polypeptides and/or sequences disclosed herein, such as SEQ ID NO:21, or combinations thereof to a cell, an assay, a subject or a sample from a subject, and detecting the activity or location of the detectable signal, or determining a change in an cellular activity, function or amount of cellular components.

The present invention comprises methods and compositions for modulating receptor binding or activity. The present invention comprises methods for modulating activity of receptor phosphotyrosine (pTyr) peptide motifs, comprising, providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a cell comprising a receptor, to an assay comprising a receptor, or to a composition comprising a receptor, and determining the activity or the binding characteristics of the receptor, or second messengers or cellular components associated with the receptor. The method may comprise inhibition of receptor binding or activity.

The present invention comprises methods and compositions for modulating binding of pTyr peptides to Stat proteins. The present invention comprises methods of modulating binding of pTyr peptides to Stat3 or Stat SH2 domain, comprising, providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a composition comprising pTyr peptides, or to cells or assays comprising pTyr peptides, and monitoring or determining the binding of pTyr peptides. pTyr peptides include, but are not limited to, native pTyr peptide, PpYLKTK, cognate pTyr peptides, native IL-6R/gp-130 derived peptide, GpYLPQTV-NH2, the Stat3 phosphopeptide, pY705Stat3, or the EGFR motif pY1068EGFR. The method may comprise inhibition of binding of pTyr peptides to Stat proteins.

The present invention comprises methods and compositions for modulating phosphorylation of a Stat monomer. The present invention comprises methods of inhibiting phosphorylation of at least one Stat monomer comprising providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a composition comprising at least one Stat monomer, or cells or an assay comprising at least one Stat monomer, and monitoring or determining phosphorylation of Stat monomers. Stat monomers include, but are not limited to, Stat3, Stat5, or other known Stat proteins. The method may comprise inhibition of phosphorylation of a Stat monomer.

The present invention comprises methods and compositions for modulating Stat3 phosphorylation by cellular kinases. The present invention comprises methods for inhibiting phosphorylation of Stat3 by cellular kinases comprising growth factor receptor tyrosine kinases, Janus kinases (Jaks), and/or the Src family kinases, comprising, providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a subject, to a cell, to a composition, to an assay, to a subject, to a sample from a subject or to a sample comprising such cellular kinases, and determining a change of phosphorylation of Stat3 proteins. The method may comprise inhibition of Stat3 phosphorylation.

The present invention comprises methods and compositions for modulating dimerization of two Stat monomers. A method comprises providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a cell, to a composition, to an assay, to a subject, to a sample from a subject or to a sample comprising Stat monomers, and determining a change in the rate of formation of, or number of, Stat dimers. The method may comprise Stat monomers wherein at least one monomer is Stat3, wherein at least one monomer is Stat 5, wherein at least on monomer is a Stat monomer. The method may comprise Stat monomers wherein both monomers are Stat3, wherein both monomers are Stat 5, wherein both monomers are Stat monomers. The method may comprise inhibition of Stat3 monomer dimerization.

The present invention comprises methods and compositions for competing with binding sites for Stat3, comprising, providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a cell, to a composition, to an assay, to a subject, to a sample from a subject or to a sample comprising receptors, small molecules, nucleic acids, cellular components, antibodies, or other binding partners for Stat3 or binding partners to which Stat3 binds, and determining the level or amount of binding, or a change in binding when the Stat3 SH2 domain mimicking polypeptide comprising SPI, or polypeptides comprising SEQ ID NO:1, or other polypeptides and/or sequences disclosed herein, such as SEQ ID NO:21, or combinations thereof are not present. For example, changes may be seen in cellular Stat3 phosphorylation, DNA binding by Stat3, or transcriptional activities by Stat3.

The present invention comprises methods and compositions for modulating Stat3 activation, comprising providing a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a cell, to a composition, to an assay, to a subject, to a sample from a subject or to a sample, and determining a change in Stat3 activation. Such modulation may occur in vitro, in vivo or in silico.

The present invention comprises methods and compositions for modulating growth or replication in a target cell, or inducing apoptosis in a target cell having aberrant or constitutive expression of Stat3, comprising contacting the target cell with a Stat3 SH2 domain mimicking polypeptide comprising SPI, or polypeptides comprising SEQ ID NO:1, or other polypeptides and/or sequences disclosed herein, such as SEQ ID NO:21, or combinations thereof, to a target cell, to a composition, to an assay, to a subject, to a sample from a subject or to a sample. A method may comprise determining a change in the target cell after the contacting.

The present invention comprises methods and compositions for modulating inflammation in a subject, comprising, providing to a subject with an inflammatory related condition, a composition comprising an effective amount of a Stat3 SH2 domain mimicking polypeptide comprising SPI, or polypeptides comprising SEQ ID NO:1, or other polypeptides and/or sequences disclosed herein, such as SEQ ID NO:21, or combinations thereof. The composition may be provided to a cell, to a composition, to an assay, to a subject, or to a sample from a subject. Inflammatory-related conditions include, but are not limited to, conditions associated with C reactive protein, acne vulgaris, asthma, automimmune diseases, chronic prostatitis, glomerulonephritis, hypersensitivities, inflammatory bowel diseases, pelvic inflammatrou disease, reperfusion injury, rheumatoid arthritis, sarcoidosis, transplant rejection, vasculitis, interstitial cystitis, atherosclerosis, allergies, myopathies, leucocyte defects, responses to pharmacological agents, and cancer.

The present invention comprises methods and compositions for modulating Stat production in a subject with an aberrant level of Stat protein, such as a subject with an inflammatory related condition. The present invention comprises methods of treating an inflammatory related condition in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in the inflammatory related condition, or other changes. Methods may further comprise administering anti-inflammatory agents in conjunction, at the same time, following or sequentially, with treatment of the inflammatory related condition with SH2 domain mimicking polypeptide compositions disclosed herein.

The present invention comprises methods and compositions for diagnosing an inflammatory related condition in a subject by determining the presence or amount of an aberrant level of Stat protein. The present invention comprises methods of detecting aberrantly produced Stat3 in at least one cell of a subject. The method may comprise detectably labeled SPI, or polypeptides comprising SEQ ID NO:1, or other polypeptides and/or sequences disclosed herein, such as SEQ ID NO:21, or combinations thereof. The method may comprise use of an antibody or fragment thereof to SPI, or polypeptides comprising SEQ ID NO:1, or other polypeptides and/or sequences disclosed herein, or combinations thereof.

The present invention comprises methods and compositions for prognosis of an inflammatory related condition in a subject by determining the presence or amount of an aberrant level of Stat protein. The present invention comprises methods of detecting aberrantly produced Stat3 in at least one cell of a subject. The method may comprise detectably labeled SPI, or polypeptides comprising SEQ ID NO:1, or other polypeptides and/or sequences disclosed herein, such as SEQ ID NO:21, or combinations thereof. The method may comprise use of an antibody or fragment thereof to SPI, or polypeptides comprising SEQ ID NO:1, or other polypeptides and/or sequences disclosed herein, such as SEQ ID NO:21, or combinations thereof.

The present invention comprises methods and compositions for determining effectiveness of anti-inflammatory agent treatment in a subject by determining the presence of, or amount of a change in, an aberrant level of Stat protein, during or after a course of treatment of an inflammatory related condition, such as with an anti-inflammatory therapeutic agent or treatment with a composition comprising SPI, or polypeptides comprising SEQ ID NO:1, or other polypeptides and/or sequences disclosed herein, such as SEQ ID NO:21, or combinations thereof. The present invention comprises methods of detecting aberrantly produced Stat3 in at least one cell of a subject, comprising, determining the level of activity or amount of Stat3 in at least one cell from a treated subject. The method may comprise detectably labeled SPI, or polypeptides comprising SEQ ID NO:1, or other polypeptides and/or sequences disclosed herein, such as SEQ ID NO:21, or combinations thereof. The method may comprise use of an antibody or fragment thereof to SPI, or polypeptides comprising SEQ ID NO:1, or other polypeptides and/or sequences disclosed herein, or combinations thereof.

The present invention comprises methods and compositions for modulating immune responses in a subject. The present invention comprises methods of treating an immune response in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in the immune response, or other changes. Methods may further comprise administering other therapeutic agents in conjunction, at the same time, following or sequentially, with treatment of the immune response with SH2 domain mimicking polypeptide compositions disclosed herein. The composition may be provided to a cell, to a composition, to an assay, to a subject, or to a sample from a subject.

The present invention comprises methods and compositions for modulating gene expression in a subject, in an assay, in a cell, or a sample from a subject. For example, the present invention comprises methods of modulating gene expression in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in gene expression, or other changes. Methods may further comprise administering therapeutic agents in conjunction, at the same time, following or sequentially, with modulating gene expression with SH2 domain mimicking polypeptide compositions disclosed herein.

The present invention comprises methods and compositions modulating cellular development in a subject, in an assay, in a cell, or a sample from a subject. For example, the present invention comprises methods of modulating cellular development in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in the cellular development, or other changes. Methods may further comprise administering therapeutic agents in conjunction, at the same time, following or sequentially, with modulating cellular development with SH2 domain mimicking polypeptide compositions disclosed herein.

The present invention comprises methods and compositions for modulating DNA transcription in a subject, in an assay, in a cell, or a sample from a subject. For example, the present invention comprises methods of modulating DNA transcription in a subject, comprising, administering to the subject an effective amount of a composition comprising SPI, or polypeptides comprising SEQ ID NO:1, or other polypeptides and/or sequences disclosed herein, such as SEQ ID NO:21, or combinations thereof. The method may comprise determining a change in the level of Stat proteins, a change in DNA transcription, or other changes. Methods may further comprise administering therapeutic agents in conjunction, at the same time, following or sequentially, with modulating DNA transcription with SH2 domain mimicking polypeptide compositions disclosed herein.

It will be appreciated by those skilled in the art that the disclosed polypeptides and nucleic acids as well as the polypeptide and nucleic acid sequences identified from any subject or patient can be stored, recorded, and manipulated on any medium that can be read and accessed by a computer. The disclosed methods can be performed in silico. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000, 5000, 10,000, or more polypeptides or nucleic acids of the invention or polypeptide sequences or nucleic acid sequences identified from any subject or patient.

Thus, provided herein is a computer system comprising a database including records for polypeptides comprising SEQ ID NO:1 and nucleic acids comprising the sequence encoding SEQ ID NO:1. Disclosed herein is a computer system comprising a database including records for polypeptides comprising variants of SEQ ID NO:1 and nucleic acids comprising the sequences encoding variants of SEQ ID NO:1. Disclosed herein is a computer system comprising a database including records for polypeptides comprising variants of SEQ ID NO:21 and nucleic acids comprising the sequences encoding variants of SEQ ID NO:21.

Computer readable medium include magnetically readable media, optically readable media, electronically readable media and magnetic/optical media. For example, the computer readable medium may be a hard disc, a floppy disc, a magnetic tape, CD-ROM, DVD, RAM, or ROM as well as other types of other media known to those skilled in the art.

Aspects of the present invention include systems, particularly computer systems which contain the sequence information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data of the disclosed compositions including, but not limited to, the disclosed polypeptides and nucleic acids.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In an aspect, the computer system includes a processor connected to a bus which is connected to a main memory, preferably implemented as RAM, and one or more data storage devices, such as a hard drive and/or other computer readable media having data recorded thereon. In an aspect, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In an aspect, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the nucleotide sequences of the nucleic acids of the invention (such as search tools, compare tools, modeling tools, etc.) may reside in main memory during execution.

In an aspect, the computer system comprises a sequence comparer for comparing polypeptide and nucleic acid sequences stored on a computer readable medium to another test sequence stored on a computer readable medium. A “sequence comparer” refers to one or more programs that are implemented on the computer system to compare a nucleotide sequence with other nucleotide sequences and to compare a polypeptide with other polypeptides.

Accordingly, an aspect of the present invention is a computer system comprising a processor, a data storage device having stored thereon a polypeptide or nucleic acid of the invention, a data storage device having retrievably stored thereon reference polypeptide or nucleotide sequences to be compared with test or sample sequences and a sequence comparer for conducting the comparison. The sequence comparer may indicate a homology level between the sequences compared or identify a difference between two ore more sequences. For example, a polypeptide comprising SEQ ID NO:1, or SEQ ID NO:21, or any fragment thereof can be compared with a test sequence from a subject or patient to determine if the test sequence is the same as the reference sequence.

Diagnostic methods or prognostic methods of the present invention comprises examining a cellular sample or medium by means of an assay, such by an assay including an effective amount of an binding partner to a peptide, such as an antibody, an affinity-purified polyclonal antibody, or a mAb (monoclonal antibody). The binding of the disclosed polypeptides, such as a polypeptide comprising SEQ ID NO:1 or SEQ ID NO:21, to Stat3 can be detected using routine methods, such as immunodetection methods, that do not disturb protein binding. The methods can be cell-based or cell-free assays. The steps of various useful immunodetection methods have been described in the scientific literature. In the most simple and direct sense, immunoassays are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The present invention comprises methods and compositions for Stat3 SH2 mimicking polypeptides. For example, an isolated Stat3 SH2 domain mimicking polypeptide comprises an amino acid sequence as set forth in SEQ ID NO:1. An isolated Stat3 SH2 domain mimicking polypeptide comprises an amino acid sequence as set forth in SEQ ID NO:21. An isolated Stat3 SH2 domain mimicking polypeptide comprises a polypeptide with amino acid sequence FISKERERAILSTKPPGTFLLRFSESSK. An isolated Stat3 SH2 domain mimicking polypeptide comprises a polypeptide with amino acid sequence ISKERERAILSTKPP. An isolated Stat3 SH2 domain mimicking polypeptide comprises a polypeptide with an amino acid sequence having greater than 75% homology to SEQ ID NO:1. An isolated Stat3 SH2 domain mimicking polypeptide comprises a polypeptide with an amino acid sequence with greater than 75% homology to SEQ ID NO:21. An isolated Stat3 SH2 domain mimicking polypeptide comprises a polypeptide that modulates one or more of constitutive Stat3 phosphorylation, Stat3 DNA binding, Stat3 transcriptional function, or Stat3 activities in vitro or in vivo. An isolated Stat3 SH2 domain mimicking polypeptide comprises a polypeptide that is a recombinant polypeptide or a synthetic polypeptide. An isolated Stat3 SH2 domain mimicking polypeptide comprises a polypeptide that comprises modified amino acids. An isolated Stat3 SH2 domain mimicking polypeptide comprises a polypeptide that is amino-terminally modified or carboxy-terminally modified, or both. An isolated Stat3 SH2 domain mimicking polypeptide comprises a polypeptide that is labeled. An isolated Stat3 SH2 domain mimicking polypeptide comprises a polypeptide with that is membrane permeable. An isolated Stat3 SH2 domain mimicking polypeptide comprises a polypeptide that is cytoplasmic membrane or a nuclear membrane permeable.

An isolated polypeptide of the present invention comprises an amino acid sequence as set forth in SEQ ID NO:1 or SEQ ID NO:21 that binds to receptor phosphotyrosine (pTyr) peptide motifs. An isolated polypeptide of the present invention comprises a polypeptide that inhibits binding of pTyr peptide motifs to Stat3 or Stat3 SH2 domain. A pTyr peptide motif comprises native pTyr peptide, PpYLKTK, native IL-6R/gp-130 derived peptide, GpYLPQTV-NH2, the Stat3 peptide, pY705Stat3, or the EGFR motif pY1068EGFR. A receptor phosphotyrosine pTyr motif comprises pTyr peptide motifs of epidermal growth factor receptor (EGFR) or pTyr peptide motifs of IL-6 receptor. An isolated polypeptide of the present invention comprises modulates phosphorylation of a Stat monomer. An isolated polypeptide of the present invention modulates dimerization of two STAT monomers. Stat monomers comprise Stat3 monomers, at least one Stat monomer is Stat3, or at least one Stat monomer is Stat5.

The present invention comprises methods and compositions comprising a peptidomimetic of Stat3 or Stat3 SH domain, comprises a polypeptide having an amino acid sequence as set forth in SEQ ID NO:1 or SEQ ID NO:21. The present invention comprises methods and compositions comprising am isolated nucleic acid encoding a Stat3 SH2 domain mimicking polypeptide comprises an amino acid sequence as set forth in SEQ ID NO:1 or SEQ ID NO:21. The present invention comprises methods and compositions comprising a vector comprises the nucleic acid of an isolated nucleic acid encoding a Stat3 SH2 domain mimicking polypeptide comprising an amino acid sequence as set forth in SEQ ID NO:1 or SEQ ID NO:21. The present invention comprises methods and compositions comprising a host cell comprising an isolated nucleic acid encoding a Stat3 SH2 domain mimicking polypeptide comprising an amino acid sequence as set forth in SEQ ID NO:1 or SEQ ID NO:21. The present invention comprises methods and compositions comprising a host cell comprising a Stat3 SH2 domain mimicking polypeptide. The present invention comprises methods and compositions comprising a composition comprising the polypeptide having an amino acid sequence as set forth in SEQ ID NO:1 or SEQ ID NO:21 and a pharmaceutically acceptable carrier. The present invention comprises methods and compositions comprising an antibody that specifically binds to an isolated Stat3 SH2 domain mimicking polypeptide, or a fragment thereof. The present invention comprises methods and compositions comprising an antibody that specifically binds to a Stat3 SH2 domain mimicking polypeptide peptidomimetic, or a fragment thereof.

The present invention comprises methods and compositions comprising a method of monitoring chemotherapy/cancer treatment, comprising determining Stat3 levels in cells or an individual using an antibody of the present invention. As aspect of methods disclosed herein may comprise measuring an amount of a cellular component, Stat3 levels or activity of cellular components, or may comprise measuring the presence or amount of a cellular component or activity before or after a treatment or administration of an agent to at least one cell. The present invention comprises methods and compositions comprising a method of cancer diagnosis, comprising, measuring the amount of Stat3 protein in a sample from a subject, using a polypeptide comprising a Stat3 SH2 domain mimicking polypeptide, wherein an aberrant amount of Stat3 indicates the presence of cancer or inflammation. A method may comprise using a monoclonal antibody of the present invention. The present invention comprises methods and compositions comprising a method of prognosis of cancer or inflammatory related condition in a subject, comprising, measuring the amount of Stat3 protein in a sample from a subject using a polypeptide comprising a Stat3 SH2 domain mimicking polypeptide, wherein an aberrant amount of Stat3 indicates the presence of cancer or inflammation. The present invention comprises methods and compositions comprising a method of modulating aberrantly produced Stat3 in an individual, comprising, administering, to the individual an effective amount of a composition comprising a Stat3 SH2 domain mimicking polypeptide. A method may further comprise administering at least one chemotherapeutic agent, wherein the at least one chemotherapeutic agent is administered in conjunction, at the same time, following, or sequentially, with the composition. Cancers that may be treated using methods or compositions of the present invention comprise uncontrolled cellular proliferation, head and neck cancer, breast cancer, prostate cancer, renal cell cancer, melanoma cancer, ovarian cancer, lung cancer, leukemia cancer, lymphoma, multiple myeloma, pancreatic cancer, or non-small cell lung cancer. The cancer may be chemotherapy-resistant cancer.

The present invention comprises methods and compositions comprising a method for determining the effectiveness of cancer treatment or anti-inflammatory agent treatment in a subject or patient, comprising, measuring an amount of Stat3 protein produced in at least one cell of a subject after a course of treatment for cancer or inflammation, and comparing the amount to an amount of Stat3 protein produced in a cell of a subject measured before treatment for cancer or inflammation, wherein measuring comprises using a detectably labeled Stat3 SH2 domain mimicking polypeptide, wherein measuring an aberrant level of Stat3 protein after treatment indicates treatment has not been effective. The present invention comprises methods and compositions comprising a method for screening for a Stat3 inhibitor, comprising, (a) providing a Stat3 SH2 domain mimicking polypeptide, (b) providing a test compound, and (c) assaying binding of the test compound to the polypeptide of step (a), wherein if the test compound binds to the polypeptide, the test compound is a Stat3 inhibitor. The present invention comprises methods and compositions comprising a method for measuring Stat3 protein levels, comprising, using an antibody to a Stat3 SH2 domain mimicking polypeptide in an immunoassay. The present invention comprises methods and compositions comprising a method for determining cellular activities or pathways in which Stat proteins function comprising, (a) administering to at least one cell, a detectable Stat3 SH2 domain mimicking polypeptide, and (b) (1) detecting the activity or location of the detectable Stat3 SH2 domain mimicking polypeptide in or on at least one cell, or (b) (2) determining a change in an cellular activity, function or amount of cellular components of at least one cell. The present invention comprises methods and compositions comprising a method of claim 45, wherein the detectable Stat3 SH2 domain mimicking polypeptide comprises a labeled polypeptide having an amino acid sequence as set forth in SEQ ID NO:1 or SEQ ID NO:21. The present invention comprises methods and compositions comprising a method of modulating binding or activity of receptor phosphotyrosine (pTyr) peptide motifs, comprising, (a) providing a Stat3 SH2 domain mimicking polypeptide to at least one cell comprising a receptor comprising a phosphotyrosine (pTyr) peptide motif The present invention comprises methods and compositions comprising a method for modulating phosphorylation of a Stat monomer, comprising, providing a Stat3 SH2 domain mimicking polypeptide to a composition or at least one cell, comprising at least one Stat monomer, wherein Stat monomers comprise Stat3, Stat5, or other Stat monomers.

The present invention comprises methods and compositions comprising a method for modulating dimerization of Stat monomers, comprising, providing a Stat3 SH2 domain mimicking polypeptide to a composition or at least one cell comprising Stat monomers. The present invention comprises methods and compositions comprising a method of competing with binding sites for Stat3, comprising, providing a Stat3 SH2 domain mimicking polypeptide to at least one cell comprising a binding partner for Stat3. A binding partner for Stat3 includes but is not limited to a Stat3 SH2 domain mimicking polypeptide, SPI, a polypeptide comprising SEQ ID NO:1, a polypeptide of SEQ ID NO:21, a receptor or peptide that binds to Stat3, a proteomimetic described herein, proteomimetics that bind to Stat3, pTyr peptide motifs, antibodies that bind to Stat 3, nucleic acids that bind to Stat3, and other known cellular or synthetic entities that selectively bind to Stat3 or a Stat3 SH2 domain mimicking polypeptide. A method may comprise providing a Stat3 SH2 domain mimicking polypeptide binding to a binding partner and measurement of changes in cellular Stat3 phosphorylation, DNA binding by Stat3 or transcriptional activities by Stat3.

The present invention comprises methods and compositions comprising a method for modulating Stat3 activation, comprising, providing a Stat3 SH2 domain mimicking polypeptide to at least one cell. Stat3 activation may be modulated in vitro, in vivo or in silico. Methods of the present invention may comprise steps performed in vitro, in vivo or in silico. The present invention comprises methods and compositions comprising a method for modulating growth or replication in a target cell, or inducing apoptosis in a target cell having aberrant or constitutive expression of Stat3, comprising, contacting the target cell with a Stat3 SH2 domain mimicking polypeptide. The present invention comprises methods and compositions comprising a method for modulating inflammation in a subject, comprising, providing to a subject with an inflammatory related condition, a composition comprising an effective amount of a polypeptide, wherein the polypeptide is a Stat3 SH2 domain mimicking polypeptide, SPI, a polypeptide comprising SEQ ID NO:1, or a polypeptide of SEQ ID NO:21. Inflammatory-related conditions comprise conditions associated with C reactive protein, acne vulgaris, asthma, automimmune diseases, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, interstitial cystitis, atherosclerosis, allergies, myopathies, leucocyte defects, responses to pharmacological agents, and cancer.

The present invention comprises methods and compositions comprising a method of modulating Stat3 production in a subject with an aberrant level of Stat3 protein, comprising, administering to the subject an effective amount of a composition comprising a Stat3 SH2 domain mimicking polypeptide, SPI, a polypeptide comprising SEQ ID NO:1, a polypeptide comprising SEQ ID NO:21. A method may comprise measuring or determining a change in the level of Stat proteins, a change in the inflammatory related condition, or other changes. The present invention comprises methods and compositions comprising a method of detecting aberrantly produced Stat3 in at least one cell of a subject, comprising, measuring the presence or amount of an aberrant level of Stat protein using an antibody to a Stat3 SH2 domain mimicking polypeptide, SPI, a polypeptide comprising SEQ ID NO:1, or a polypeptide comprising SEQ ID NO:21. The present invention comprises methods and compositions comprising a method of detecting aberrantly produced Stat3 in at least one cell of a subject, comprising, determining the presence or amount of an aberrant level of Stat protein using a Stat3 SH2 domain mimicking polypeptide, SPI, a polypeptide comprising SEQ ID NO:1, or a polypeptide comprising SEQ ID NO:21, wherein the polypeptide is detectably labeled. The present invention comprises methods and compositions comprising a method for modulating immune responses in a subject, comprising, administering to the subject an effective amount of a composition comprising a Stat3 SH2 domain mimicking polypeptide, SPI, a polypeptide comprising SEQ ID NO:1, or a polypeptide comprising SEQ ID NO:21. A method may further comprise administering at least one therapeutic agent in conjunction, at the same time, following or sequentially, with modulating immune response with the polypeptide. The present invention comprises methods and compositions comprising a method for modulating gene expression, comprising, administering to at least one cell, an effective amount of a composition comprising a Stat3 SH2 domain mimicking polypeptide, SPI, a polypeptide comprising SEQ ID NO:1, or a polypeptide comprising SEQ ID NO:21. The present invention comprises methods and compositions comprising a method of modulating cellular development, comprising, administering to at least one cell, an effective amount of a composition comprising a Stat3 SH2 domain mimicking polypeptide, SPI, a polypeptide comprising SEQ ID NO:1, or a polypeptide comprising SEQ ID NO:21. A method may further comprise measuring a change in the level of Stat proteins, a change in cellular development, or other cellular changes. The present invention comprises methods and compositions comprising a method for modulating DNA, comprising, administering to at least one cell, an effective amount of a composition comprising a Stat3 SH2 domain mimicking polypeptide, SPI, a polypeptide comprising SEQ ID NO:1, or a polypeptide comprising SEQ ID NO:21. A method may further comprise measuring a change in the level of Stat proteins, a change in DNA transcription, or other changes.

The terminology used herein is for the purpose of describing particular aspects only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, an aspect includes from the one particular value and/or to the other particular value. Similarly, when values are expressed as approximations, by use of the antecedent “about,” it will be understood that the particular value forms an aspect. It will be further understood that the endpoints of each of the ranges are significant both in relation to the other endpoint, and independently of the other endpoint.

The amino acid abbreviations used herein are conventional one letter codes for the amino acids and are expressed as follows: Ala or A for Alanine; Arg or R for Arginine; Asn or N for Asparagine; Asp or D for Aspartic acid (Aspartate); Cys or C for Cysteine; Gln or Q for Glutamine; Glu or E for Glutamic acid (Glutamate); Gly or G for Glycine; H is or H for Histidine; Ile or I for Isoleucine; Leu or L for Leucine; Lys or K for Lysine; Met or M for Methionine; Phe or F for Phenylalanine; Pro or P for Proline; Ser or S for Serine; Thr or T for Threonine; Trp or W for Tryptophan; Tyr or Y for Tyrosine; Val or V for Valine; Asx or B for Aspartic acid or Asparagine; and Glx or Z for Glutamine or Glutamic acid.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide.

As used herein, “cognate” refers to an entity of a same or a similar nature.

As used herein, the term “amino acid sequence” refers to a list of abbreviations, letters, characters or words representing amino acid residues.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One aspect of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “reverse analog” or “reverse sequence” refers to a peptide having the reverse amino acid sequence as another reference peptide. For example, if one peptide has the amino acid sequence ABCDE, its reverse analog or a peptide having its reverse sequence is as follows: EDCBA.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100 percent, or more, or any amount of promotion in between compared to native or control levels. In an aspect, the increase or promotion is 10-20, 20-30, 30-40, 40-50, 50-60, 60-70, 70-80, 80-90, or 90-100 percent as compared to native or control levels. In an aspect, the increase or promotion is 0-25, 25-50, 50-75, or 75-100 percent, or more, such as 200, 300, 500, or 1000 percent more as compared to native or control levels. In an aspect, the increase or promotion can be greater than 100 percent as compared to native or control levels, such as 100, 150, 200, 250, 300, 350, 400, 450, 500 percent or more as compared to the native or control levels.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

The term “sample” can refer to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### 1. Computer-Aided Design of SPI as a Molecular Probe and Stat3 Inhibitor

Close structural analysis of the lowest Genetic Optimization for Ligand Docking (GOLD) (Jones et al., 1997) conformation of the native pTyr peptide, PpYLKTK bound within the Stat3 SH2 domain (Siddiquee et al., 2007), per the X-ray crystal structure of Stat3β homodimer (Becker et al., 1998), showed significant complementary interactions at the protein surface, by which a minimum SH2 domain peptide sequence was derived that retains interactions with the pTyr peptide. The lowest energy GOLD docking studies consistently showed the pTyr peptide making hydrogen bonds and electrostatic interactions with the residues, Lys591, Ser611, Ser613 and Arg609 of the SH2 domain. The SH2 domain peptide, SPI, was composed of amino acid residues 588-615, which incorporate the aforementioned residues. The spatial presentation of the 28-mer peptide (28-mer) within the context of the 3D-structure of Stat3, was examined and developed per comparative modeling methods using ModWeb (University of California San Francisco, UCSF) (Pettersen et al., 2004) with a score of 0.87. The structure of SPI was modeled using ModWeb, per the X-ray crystal of Stat3β (Becker et al., 1998), and viewed by Chimera software (UCSF) (Pettersen et al., 2004). Due to ModWeb software requirements, of a minimum of 30 amino acids, three Gly residues were added to the N-terminus of the 28-mer during the modeling, but were eliminated at the time of viewing with Chimera software.

Sequence alignment analysis of SPI with SH2 motifs from other proteins revealed high homologies, 78% and 73%, respectively, to the Stat1 and Stat4 SH2 domains, and 40-57% homologies to the other proteins evaluated, including Stat2, Stat5, Stat6, Src, Fyn, Fgr, TNS3 (Tensin-3 protein), and SH2D2A (SH2 domain protein 2A) (Table 3). Molecular modeling raised the potential that SPI retained significant 3-D structural characteristics as in the full-length Stat3 protein. Studies were conducted for the characterization of the biochemical and biophysical properties of SPI relative to Stat3, and to determine the potential to inhibit Stat3 activation and functions.

TABLE 3
Sequence homology between SPI and SH2 Motifs of Select Human Proteins

| Protein | Gene ID | Identity (%) |
|---|---|---|
| Stat3 | 6774 | 100 |
| Stat1 | 6772 | 78 |
| Stat4 | 6775 | 73 |
| Stat2 | 6773 | 57 |
| Stat6 | 6778 | 57 |
| Stat5A | 6776 | 53 |
| Stat5B | 6777 | 53 |
| Fyn | 2534 | 53 |
| Fgr | 2268 | 50 |
| Src | 6714 | 50 |
| TNS3 | 64759 | 48 |
| SH2D2A | 9047 | 44 |

### 2. SPI Interacted with Cognate pTyr Peptide Motifs and Disrupted the Binding of Stat3 or the Stat3 SH2 Domain

To provide definitive evidence of direct binding of SPI to known Stat3-binding pTyr peptide motifs and to compare to the binding of Stat3 or the Stat3 SH2 domain, biophysical studies were performed. Purified His-tagged Stat3 protein (50 μg), His-tagged Stat3 SH2 domain (30 μg), or SPI (25 μg) was immobilized (target) on a Ni-NTA or carboxy sensor chip surface for Surface Plasmon Resonance (SPR) analysis of the binding to pTyr peptides (analyte). Association and dissociation measurements were taken and the affinities were determined using Qdat software. The known Stat3-binding pTyr peptide motifs, PpYLKTK (pY705 Stat3 involved in Stat3: Stat3 and Stat1:Stat3 dimerization events) were considered. GpYIKTE (pY701Stat1 involved in Stat1:Stat1 and Stat1:Stat3 dimerization events). PEpYINQS and PVpYHNQP (pY1068EGFR and pY1086EGFR, respectively), and GpYLPQTV (IL-6R/gp 130), and the probable binding motif, GpYVKJPQ (pY694Stat5) (pTyr peptide sequences were derived from the NCB1 protein database), and the previously reported Stat3 dimerization inhibitor and the SH2 domain antagonist, S31-201 (Siddiquee et al., 2007).

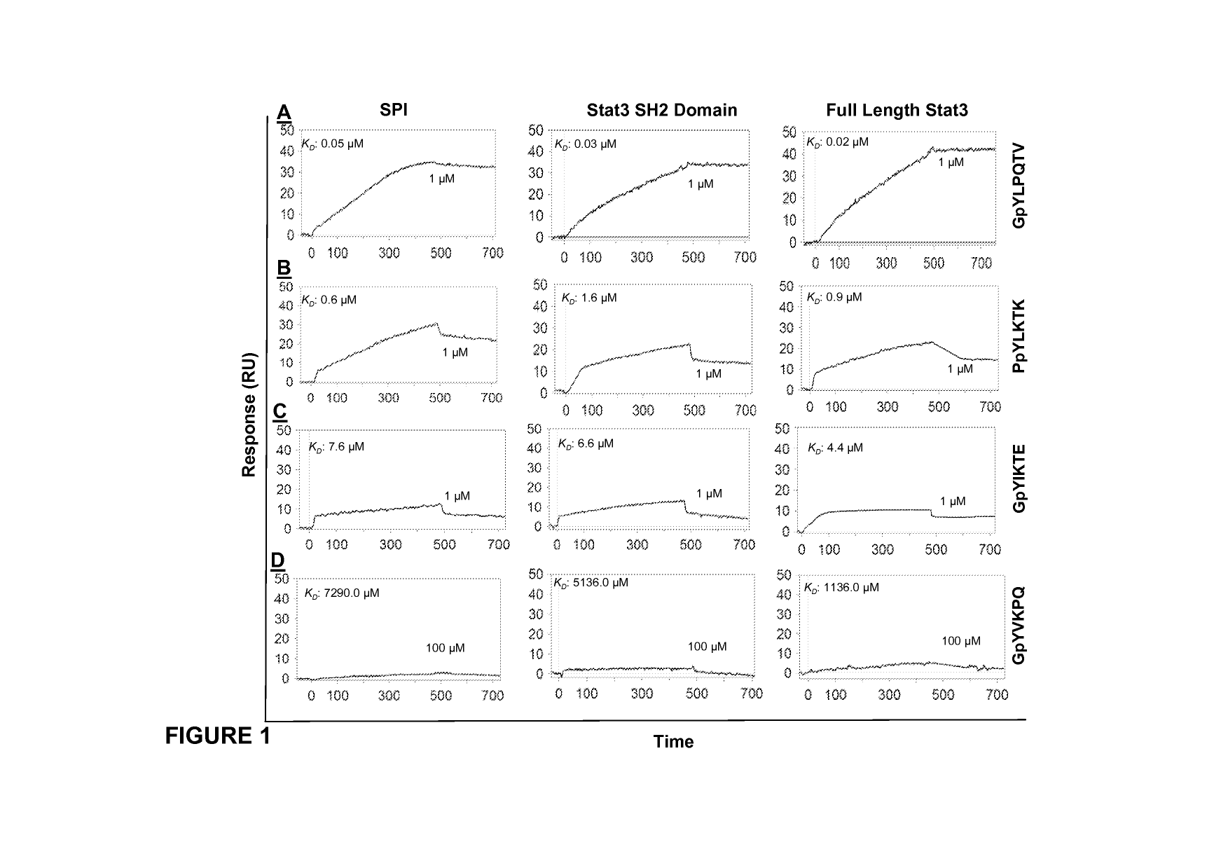

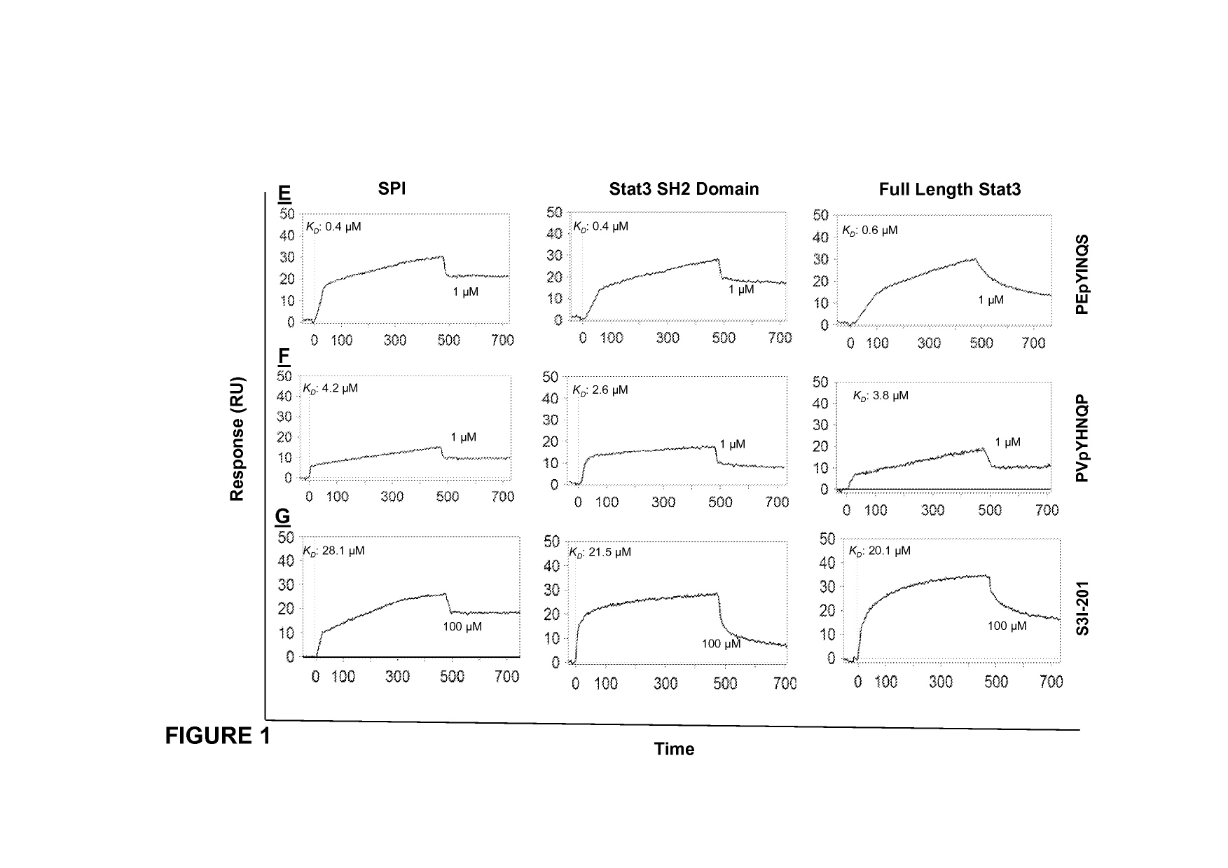

The interactions with the aforementioned pTyr peptides (analytes) were comparable for the three targets (SPI, Stat3 SH2 domain, and full length Stat3). Specifically, the interactions with the gp130-derived peptide, GpYLPQTV, were of the highest affinity, with K_{D} values of 50.0, 30.0, and 20.0 nM, respectively, for SPI, Stat3 SH2 domain, and full-length Stat3 (FIG. 1A, GpYLPQTV). Relative to these affinities, the interactions with the Stat3-derived pTyr705 peptide, PpYLKTK were 13 to 100-fold weaker, with K_{D} values of 0.6, 1.6, and 0.9 μM, for SPI, Stat3 SH2 domain, and full length Stat3, respectively (FIG. 1B, PpYLKTK), as were the interactions with the pY1068 of EGFR, PEpYINQS, with K_{D} values of 0.4, 0.4, and 0.6 μM, respectively, for SPI, Stat3 SH2 domain, and Stat3 (FIG. 1E, PEpYINQS). Even much weaker were the interactions of the three targets with the pY1086 peptide of EGFR, PVpYHNQP, of 4.2, 2.6, and 3.8 μM, respectively, for SPI, Stat3 SH2 domain, and full length Stat3 (FIG. 1F, PVpYHNQP). These data indicated that the two known Stat3-binding EGFR pTyr motifs, pY1086EGFR (PEpYINQS, FIG. 1E) and pY1086EGFR (PVpYHNQP, FIG. 1F), Stat3 and SPI display differential binding. These data also demonstrated the relative weaker binding to the Stat1 pY701 peptide, GpYIKTE (FIG. 1C), with K_{D} values 7.6, 6.6, and 4.4 μM, for SPI, Stat3 SH2 domain, and Stat3, which compared to the binding to Stat3 pY705 peptide (PpYLKTK, FIG. 1B) represented a 3 to 13-fold difference in affinity, despite there being 78% sequence homology between the Stat3 and Stat1 SH2 domains (Table 3) and that when activated concurrently, Stat1 and Stat3 engaged in a heterodimer formation. The binding of full length Stat3 (or Stat3 SH2 domain) or SPI to the Stat1 pY701 peptide was weaker compared to their binding to the pTyr peptides derived from the IL-6R/gp130, pY705Stat3, or the pY1068EGFR.

The data, which are representative of three (3) independent determinations, indicated SPI and monomeric pStat3 have preference for pStat3 monomer over pStat1 monomer. SPR analysis similarly showed SPI, Stat3 SH2 domain, and full length Stat3 interact with the small-molecule, SH2 domain antagonist, S31-201, with K_{D} values of 28.1, 21.5, and 20.1 μM, respectively (FIG. 1G, S3I-201). Although weaker, the interactions with S31-201 exhibited similar characteristics for SPI and Stat3 (or Stat3 SH2 domain). By contrast, data show that SPI, Stat3 SH2 domain, and full length Stat3 interacted poorly with the pTyr peptide of Stat5, GpYVKPQ (FIG. 1D), with K_{D} values of 7.3, 5.2, and 1.2 mM, respectively (FIG. 1D, GpYVKPQ). These data indicated far weaker affinities for Stat5, and also indicate that SPI, like pStat3, was more likely to interact with pStat3 than pStat5. Furthermore, the data indicated that interactions with pStat5 weee rather a low probability, given the millimolar affinities.

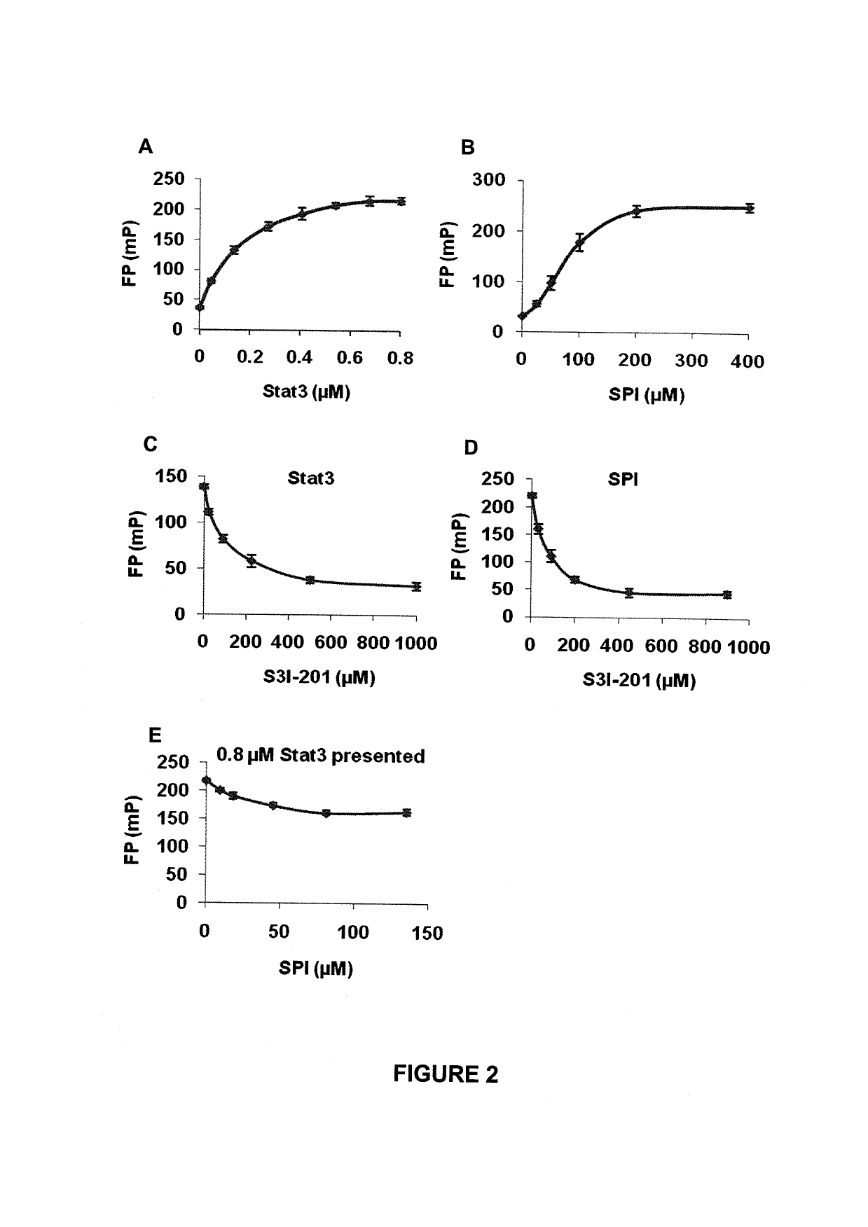

The studies demonstrated that SPI interacts with cognate pTyr peptide motifs recognized by Stat3 (or Stat3 SH2 domain). The interaction with SPI blocked the binding of Stat3 to its cognate pTyr peptide motifs. Fluorescence polarization (FP) study has previously been used to demonstrate the binding of Stat3 (or Stat3 SH2 domain) to the high-affinity peptide, GpYLPQTV-NH₂ (Ren et al., 2003; Schust et al., 2006; Zhang et al., 2010). Therefore, this assay was used to verify SPI binding to the high-affinity pTyr peptide motif and to further assess the potential that such a binding disrupted the association of Stat3 with the same peptide probe. Results of the FP assay, which utilized the 5-carboxyfluorescein-GpYLPQTV-NH₂ as a probe, showed a similar binding saturation profile, measured as fluorescence polarization signal (mP), with the increasing concentration (in μM) of purified His-Stat3 (FIG. 2A) or SPI (FIG. 2B), indicating that SPI binds similarly as Stat3 to the pTyr peptide probe.

To further evaluate the SPI:peptide probe interaction, relative to the binding of Stat3 to the same probe, the responsiveness of the interactions to the known Stat3 dimerization inhibitor, S31-201 (Siddiquee et al., 2007), were compared. Here, fixed concentrations of each of Stat3 (200 nM) and SPI (150 μM) were incubated with increasing concentrations of S31-201 prior to incubation with the 5-carboxyfluorescein-GpYLPQTV-NH₂ probe. The FP measurements were collected. Consistent with the observed similarities in the saturation curves (FIGS. 2A-2B), the profiles of the S3I-201-induced inhibition of the binding to the labeled peptide probe were similar, regardless of whether Stat3 or SPI was the ligand showed binding of Stat3 (FIG. 2C) and shows binding of SPI (FIG. 2D).

Next, the potential of SPI to compete against Stat3 by binding to the labeled pTyr peptide probe was examined To address this, aliquots of fixed concentration of Stat3 (0.8 μM) were each incubated with a different concentration of SPI, prior to incubation with the fluoresceinyl-labeled probe and subjected to FP measurements. Results showed concentration-dependent decrease in FP signal when SPI is present up to a certain concentration, when no further decreases became evident (FIG. 2E), indicating that SPI disrupted the interaction of Stat3 with the cognate pTyr peptide probe. In this context, the FP signal decreased to the levels consistent with the displacement of Stat3 and the binding of SPI, as a ligand. These data together demonstrated that SPI, like Stat3, binded to cognate pTyr peptide motifs. By this mechanism, SPI disrupted Stat3:Stat3 dimerization and the Stat3 binding to cognate pTyr peptide motifs. The FP assay based on the 5-carboxyfluorescein-GpYLPQTV-NH₂ probe and SPI was rigorously tested and validated, with a Z factor of 0.89. The data in FIG. 2A-2E are representative of four (4) independent determinations.

### 3. Intracellular Accumulation of SPI and Inhibition of Intracellular Stat3 Activation

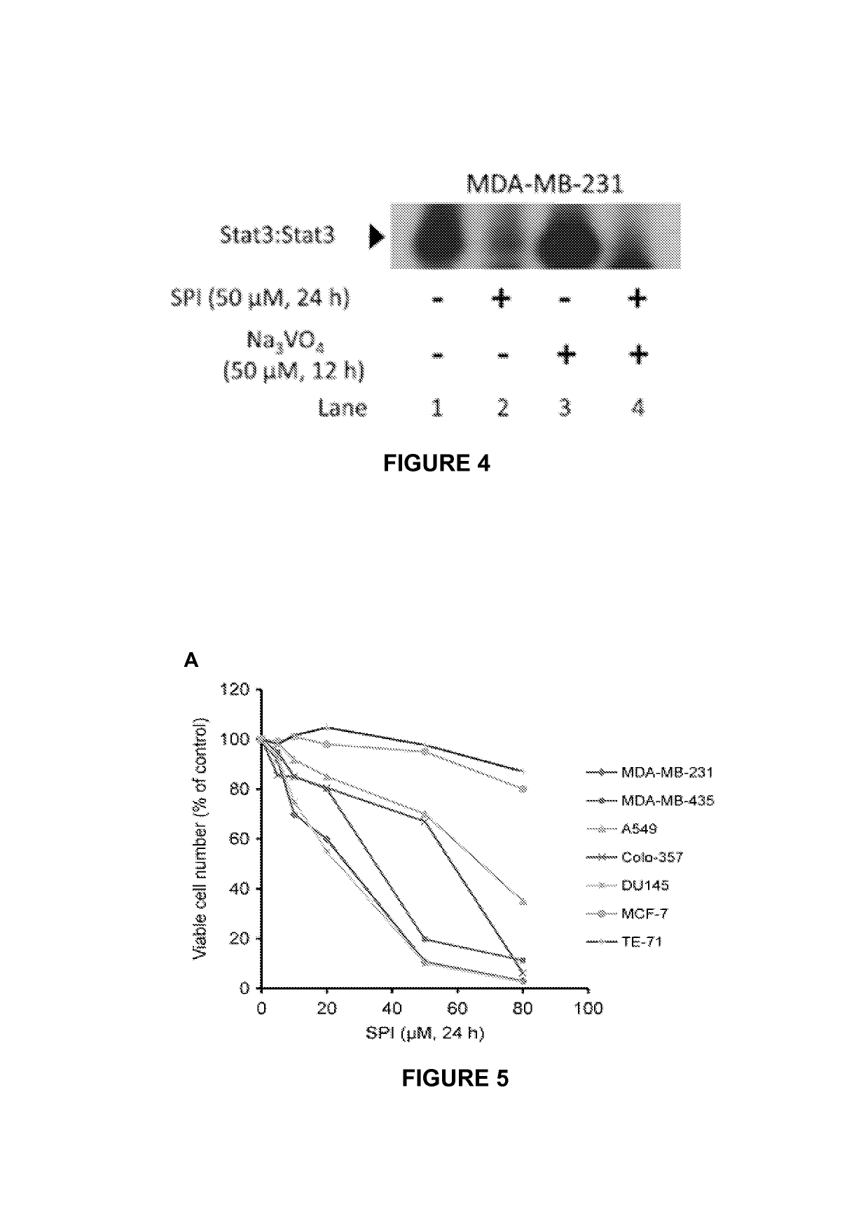

Stat3 was constitutively-activated in a variety of malignant cells, including human breast and pancreatic cancer cells (Yu et al., 2004; Yue et al., 2008; Turkson 2004). Given the effect against the binding of Stat3 (or Stat3 SH2 domain) to cognate pTyr peptide motifs, whether SPI inhibited Stat3 activation in malignant cells was evaluated. To address this, the extent of intracellular uptake of SPI was evaluated by creating a 5-carboxyfluorescein-labeled version in which the fluorescence tag was attached to the amino-terminus of SPI. Fluorescent microscopy analysis showed significant cellular uptake of SPI by a variety of cells, including the human breast cancer line, MDA-MB-231, and NIH3T3/hEGFR, following treatment with fluorescently-labeled 30 μM SPI for 6 hours (FIG. 4A). Observation under higher magnification (40×) showed a wide intracellular distribution of SPI, with nuclear accumulation, demonstrating SPI was membrane-permeable and localized in the nucleus.

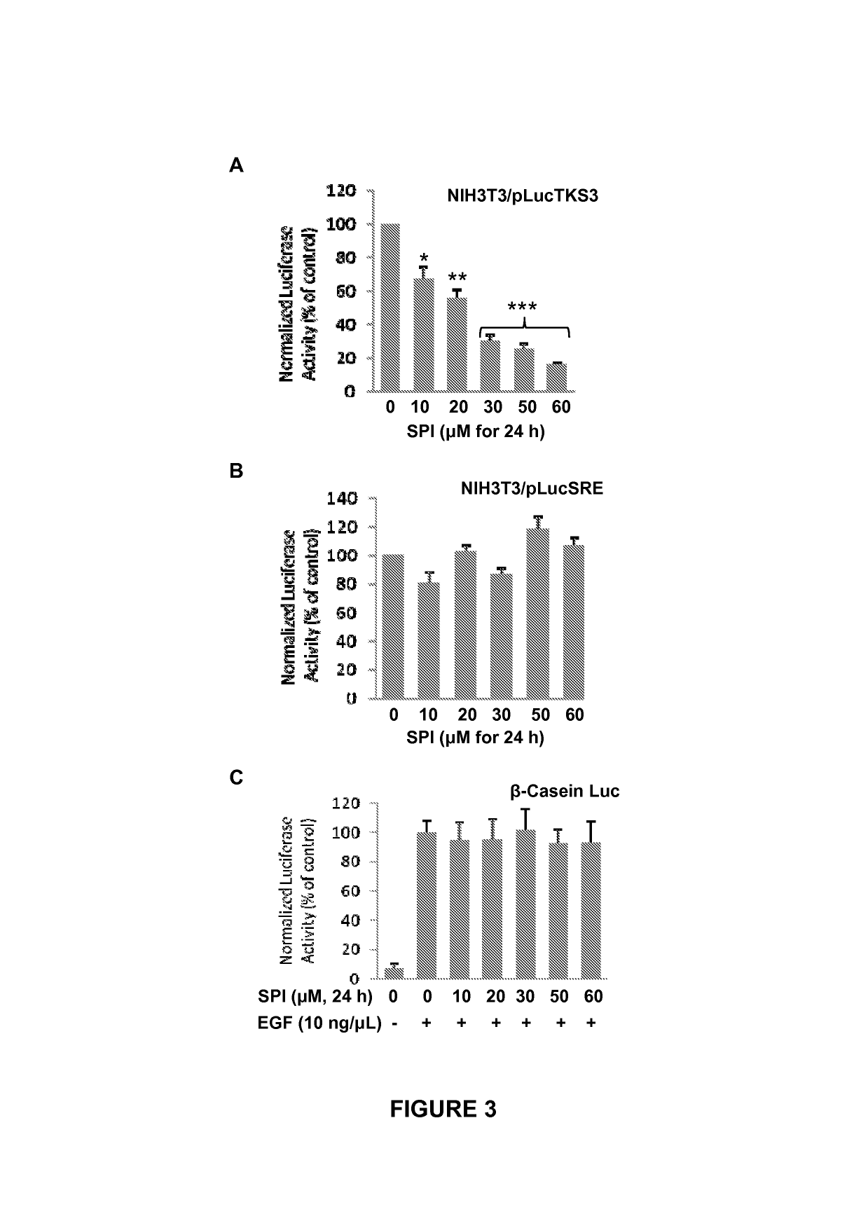

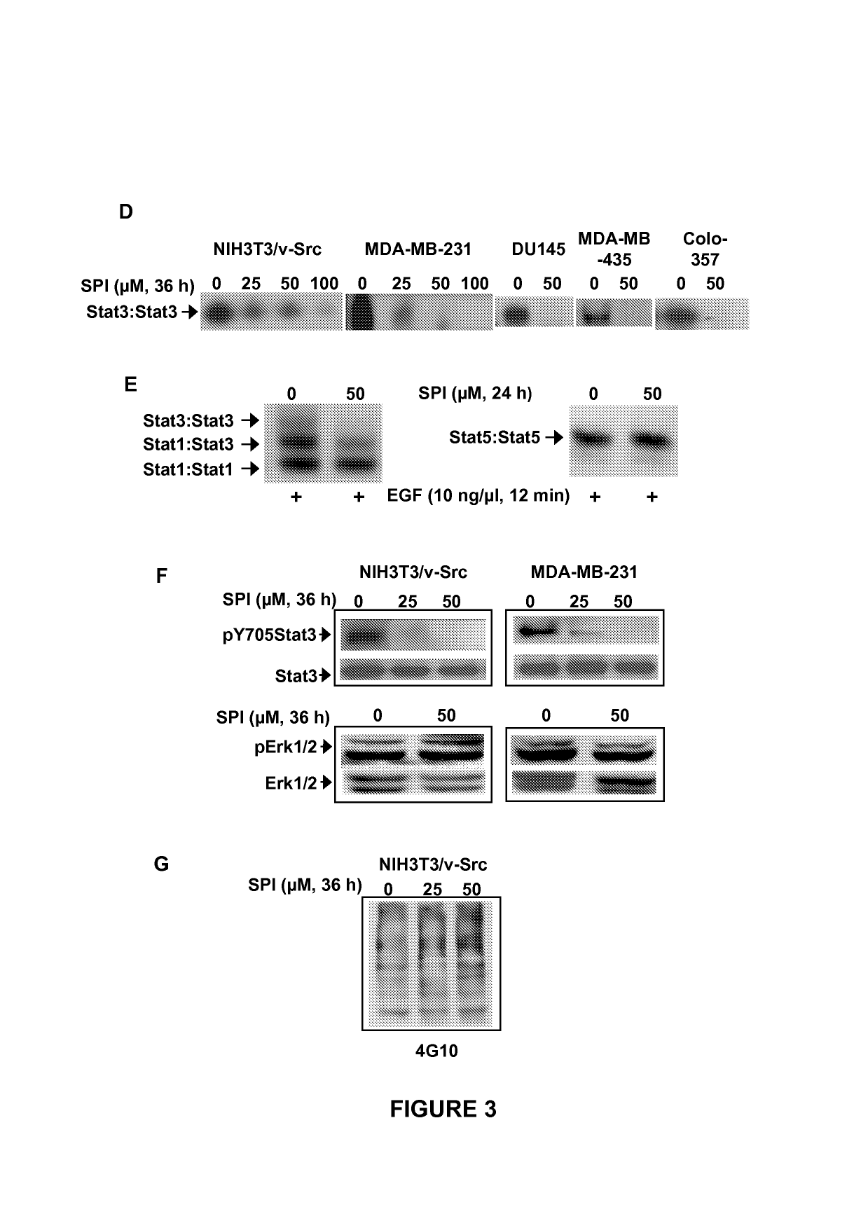

Malignant cells were then treated with SPI to assess biochemical and biological effects. Treatment of v-Src-transformed mouse fibroblasts harboring aberrant Stat3 activity (NIH3T3/v-Src/pLucTKS3) (Turkson et al., 2004; Turkson et al., 2001) and that stably over-express the Stat3-dependent luciferase reporter, pLucTKS3 (Turkson et al., 2004; Turkson et al., 2001; Turkson et al., 1999; Turkson et al., 1998) showed a dose-dependent inhibition of Stat3-mediated luciferase reporter induction (FIG. 3A), demonstrating that SPI inhibited Stat3 transcriptional activity. By contrast, a similar treatment of the v-Src-transformed mouse fibroblasts (NIH3T3/v-Src/pLucSRE) (Turkson et al., 2004; Turkson et al., 2001) that stably over-express the Stat3-independent luciferase reporter, pLucSRE (Turkson et al., 1999; Turkson et al. 1998), which was driven by the serum response element (SRE) of the c-fos promoter (FIG. 3B), or of EGF-stimulated mouse fibroblasts transiently-transfected with the Stat5-dependent β-Casein-promoter-driven luciferase (β-Casein-Luc) (Siddiquee et al., 2007) had minimal to no effect on the induction of these reporters (FIG. 3C).

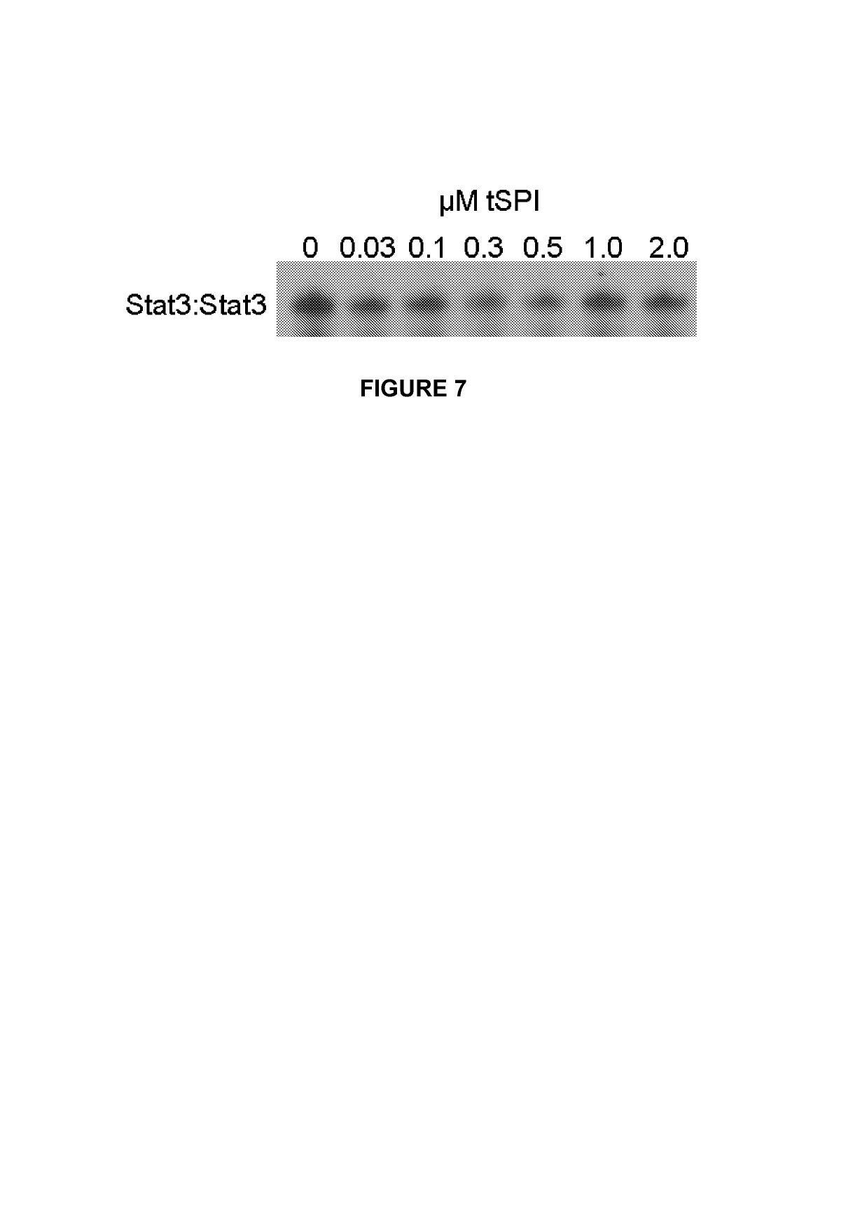

Consistent with the inhibition of Stat3 transcriptional activity, v-Src-transformed mouse fibroblasts (NIH3T3/v-Src), or the human breast (MDA-MB-231 and MDA-MB-435), prostate (DU145), or pancreatic (Colo-357) cancer cells treated with SPI showed a dose-dependent inhibition of constitutive Stat3 activation, as measured by DNA-binding activity in nuclear extract preparations using electrophoretic mobility shift assay (EMSA), with a near complete inhibition at 50 μM (FIG. 3D). The results of an experiment using a truncated version of SPI (ISKERERAILSTKPP as represented by SEQ ID NO:21) is shown in FIG. 7 (using 0 to 2 μM of a truncated version of SPI). The induction of the EGF receptor in fibroblasts activates Stat1, Stat3 and Stat5, which formed homo- and hetero-dimers, as measured by DNA-binding/EMSA analysis (FIG. 3E). Consistent with the effects on Stat3 activation, the prior treatment of mouse fibroblasts over-expressing the human EGFR (NIH3T3/hEGFR) with 50 μM SPI blocked EGF-induced Stat3 activation, measured as Stat3:Stat3:DNA complex (FIG. 3E, left panel, upper band), and suppressed the Stat1:Stat3 heterocomplex, measured as Stat1:Stat3:DNA complex (FIG. 3E, left panel, intermediate band).

By contrast, similar treatment had little or no effect on Stat1:Stat1:DNA complex (FIG. 3E), left panel, lower band) or Stat5:Stat5:DNA complex (FIG. 3E, right panel). Furthermore, immunoblotting analysis of whole-cell lysates prepared from SPI-treated NIH3T3/v-Src and MDA-MB-231 cells showed a selective suppression of pY705Stat3 (FIG. 3F, upper panel), with no repression of pErk1/2 levels (FIG. 3F, lower panel). Total Stat3 protein levels remained unchanged (FIG. 3F, upper panel) Immunoblotting analysis further showed no significant changes in the general pTyr profile of v-Src-transformed mouse fibroblasts treated with SPI (FIG. 3G). Thus, at concentrations up to 50 μM, SPI selectively inhibited constitutive Stat3 activation and transcriptional activity in malignant cells.

### 4. Biochemical Mechanism of Inhibition of Intracellular Stat3 Activation

The mode of inhibition of Stat3 activation was examined. As demonstrated by the SPR and FP studies, SPI binded to pTyr peptide motifs and disrupted Stat3:Stat3 dimerization, as observed for small-molecule or peptidomimetic dimerization disruptors of Stat3 (Turkson et al., 2004; Turkson et al., 2001). The SPR data showed that both Stat3 (or Stat3 SH2 domain) and SPI binded to the known Stat3-binding pY1068EGFR and pY1086EGFR motifs with comparable affinities. SPI, like the Stat3 SH2 domain associated with receptor pTyr motifs. The binding to receptor motifs obstructed Stat3 binding and thereby blocked de novo Stat3 phosphorylation and activation. To verify this and to determine the intracellular localization and colocalizations of SPI and EGFR, fluorescence microscopy and immunofluorescence staining with laser-scanning confocal microscopy analyses were conducted. Cells were treated with the fluorescently-labeled SPI and subjected to immunostaining for EGFR. The microscopy experiments showed that in MDA-MB-231 cells treated with fluorescently-labeled SPI, the 28-mer is widely distributed throughout cells with a nuclear localization. Moreover, SPI and EGFR were colocalized at the plasma membrane in MDA-MB-231 and in mouse fibroblasts over-expressing EGFR (NIH3T3/hEGFR) and treated with fluorescently-labeled SPI.

The observed colocalization indicated SPI associates with EGFR. To test whether SPI association with receptors competed against the binding of Stat3, and blocked de novo activation, MDA-MB-231 cells were treated with or without SPI for 8 hours prior to treatment with sodium orthovanadate (protein phosphatase inhibitor) for an additional 16 hours. Analysis by in vitro DNA-binding activity assay/EMSA of nuclear extract preparations showed that the treatment with SPI alone inhibited Stat3 DNA-binding activity (FIG. 4, lane 2, compared to lane 1). The treatment with orthovanadate alone increased Stat3 activity above the existing levels (FIG. 4, lane 3, compared to lane 1), which was in part due to the blockade of the pStat3 turnover by the inhibition of protein phosphatases.

By contrast, unlike cells treated with orthovanadate alone, in cells treated first with SPI and then with orthovanadate, Stat3 activation was completely inhibited (FIG. 4, lane 4). This inhibition can be the result of the blockade of de novo Stat3 activation (by SPI), concomitant with the physiological turnover (elimination) of pre-existing pStat3 during the period prior to the addition of orthovanadate. Thus, by the time of the orthovanadate addition, there were no residual pStat3 levels. The microscopy and the gel shift data together demonstrated that SPI associates with Stat3-binding motifs of receptors, and prevented de novo Stat3 phosphorylation.

### 5. SPI Blocked Cell Viability and Growth, and Induced Apoptosis of Malignant Cells Harboring Constitutively-Active Stat3

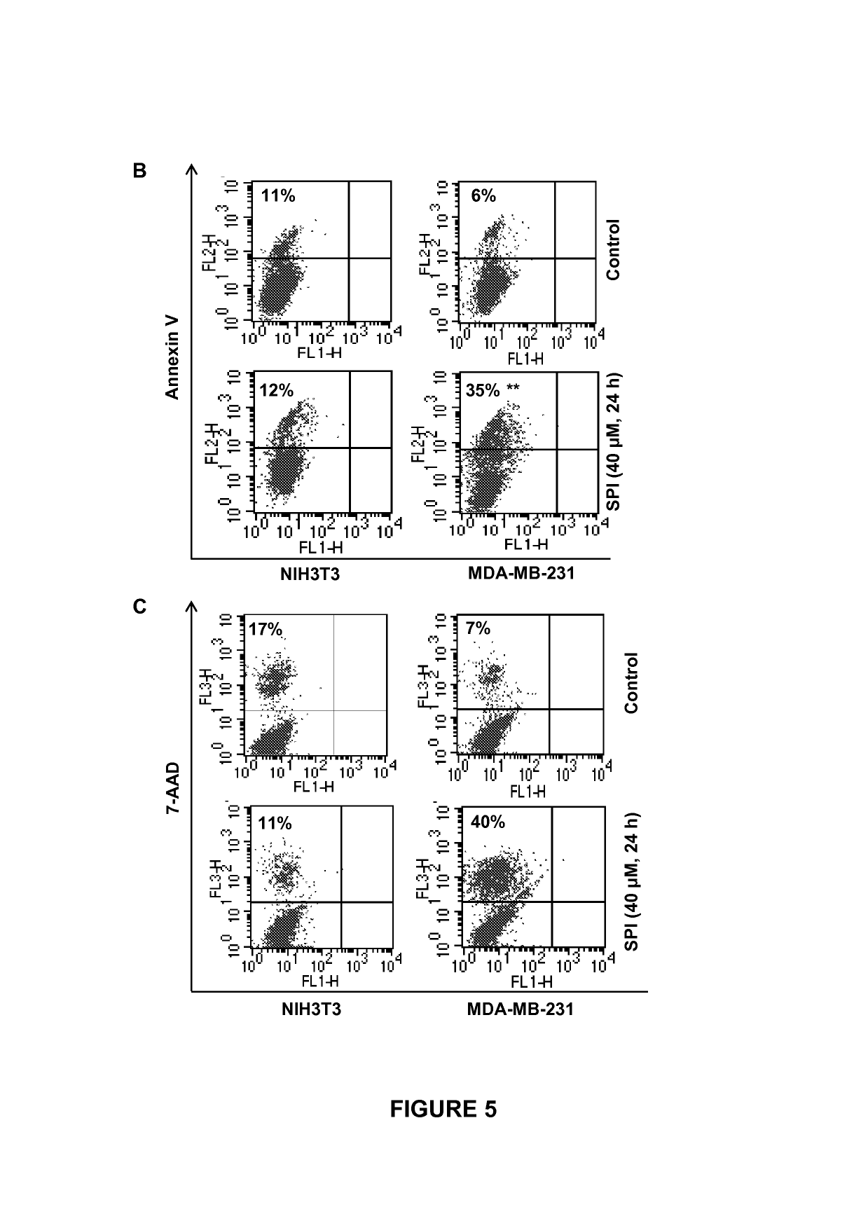

Aberrantly-active Stat3 promoted malignant cell proliferation and survival and malignant transformation (Yue et al., 2008; Turkson et al., 2004; Siddiquee et al., 2008). Whether SPI was able to selectively decrease the viability and growth of malignant cells that harbor aberrant Stat3 activity was determined. The human breast (MDA-MB-231 and MDA-MB-435), pancreatic (Colo-357), prostate (DU145), and non-small cell lung cancer (A549) lines that harbor constitutively-active Stat3 in culture were treated with or without an increasing concentration of SPI for 24 hours. The cells were visualized under phase-contrast microscope for morphology changes, or analyzed for viable cell numbers by CyQuant cell proliferation/viability kit or by trypan blue exclusion with phase-contrast microscopy. Compared to the control (DMSO-treatment), the human tumor cells harboring aberrant Stat3 activity (MDA-MB-231, MDA-MB-435, DU145, Colo-357, and A549) and treated with SPI showed significant morphology changes and had reduced viable cell numbers (FIG. 5A). The Stat3-dependent rumor cell lines showed dose-dependent decreases in viability and growth following 24 hours treatment with increasing concentration of SPI (FIG. 5A).

By contrast, the morphology, viability, and growth of cells that did not harbor aberrant Stat3 activity (normal NIH3T3, human breast cancer, MCF-7, murine thymus stromal epithelial cells, TE-71 and prostate cancer, LNCaP) were not significantly affected by similar treatment with SPI (FIG. 5A). Furthermore, cultured MDA-MB-231 cells that harbor aberrant Stat3 activity and treated with SPI for 24 hours and subjected to Annexin V binding/flow cytometry analysis showed evidence of significant apoptosis (34%), compared to DMSO-treated control (6%), while the normal NIH3T3 fibroblasts similarly treated showed little evidence of apoptosis, compared to DMSO-treated control (FIG. 5B, left panel). These data together, which represent 3-4 independent determinations, indicated that SPI selectively repressed constitutive Stat3 activation in malignant cells, and induced antitumor cell effects that were dependent on the presence of constitutively-active Stat3 in cells.

### 6. SPI Inhibits Tumor Growth

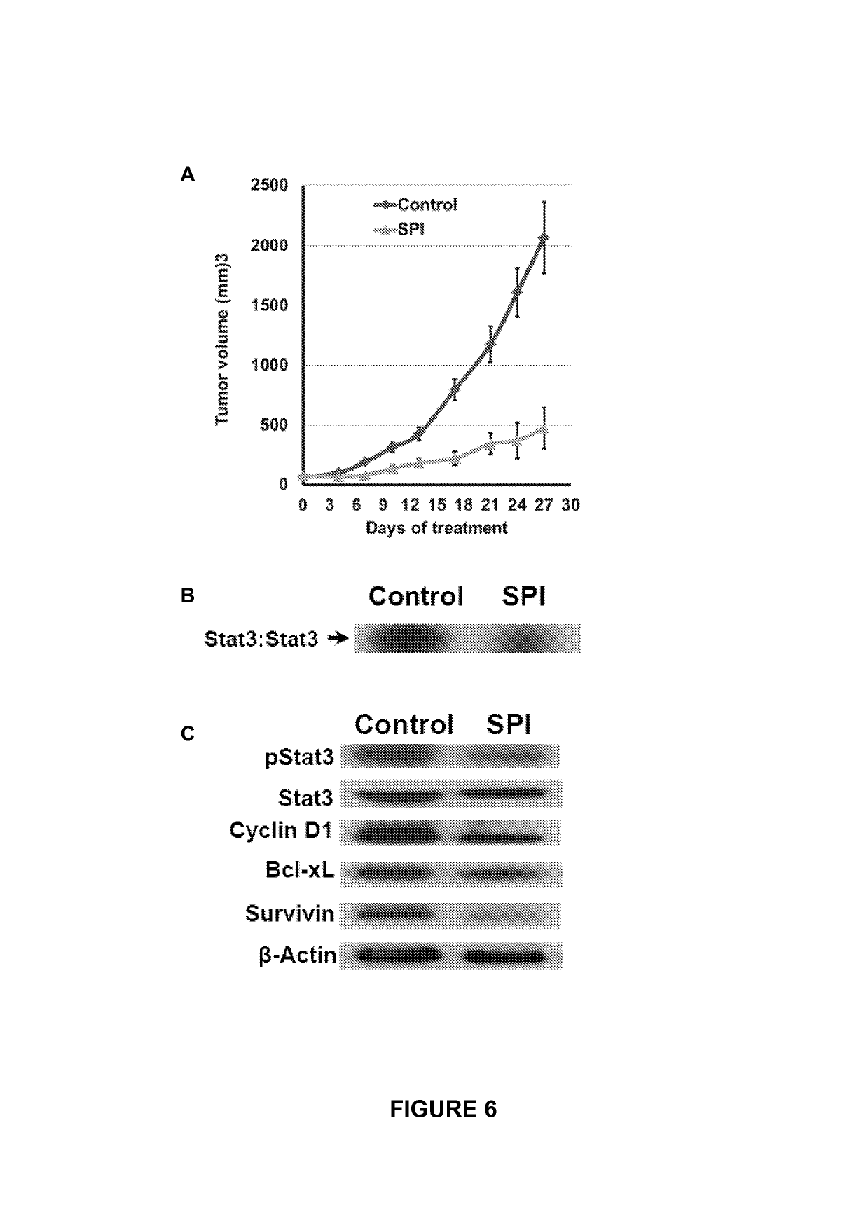

Mice bearing human breast tumors were given SPI (8 mg/kg) intravenously every 3-4 days during a 30 day period. Tumor sizes were measured and the tumor volume was calculated. As shown in FIG. 6A, the tumor bearing mice receiving SPI treatment had a tumor volume that was than the tumor volume of the control mice receiving no SPI treatment. In FIG. 6A, the value represents the mean±standard deviation (SD). FIG. 6B showed the DNA-binding activity/EMSA analysis of tumor lysates from the SPI-treated mice or the control mice (DMSO-treated) using a hSIE probe. FIG. 6C showed the western blotting analysis of lysates of equal total protein prepared from control tumor or residual tumors from SPI-treated mice using antibodies against pStat3, Stat3, Cyclin D1, Bcl-xl, Survivin, and β-actin.

### 7. Experimental Procedures

Cells and reagents. Normal mouse fibroblasts (NIH3T3) and counterparts transformed by v-Src (NIH3T3/v-Src) or overexpressing the human epidermal growth factor (EGF) receptor (NIH3T3/hEGFR), and the human breast (MDA-MB-231, MDA-MB-435, and MCF-7), pancreatic (Colo-357), prostate (DU145 and LNCaP), non-small cell lung (A549) cancer, and TE-71 mouse thymus epithelial stromal cells have all been previously reported (Turkson et al., 2001; Johnson et al., 1985; Yu et al., 1995; Garcia et al., 2001; Zhang et al., 2010; Far et al., 1989). The Stat3-dependent reporter, pLucTKS3 and the Stat3-independent reporter, pLucSRE, and the v-Src transformed mouse fibroblasts that stably express pLucTKS3 (NIH3T3/v-Src/pLucTKS3) or pLucSRE (NIH3T3/v-Src/pLucSRE), and the Stat3-independent β-Casein luciferase reporter (β-Casein-Luc) driven by the Stat5-responsive β-Casein promoter have been previously reported (Turkson et al., 2004; Turkson et al., 2001; Siddiquee et al., 2007; Turkson et al., 1999; Turkson et al., 1998). Cells were grown in Dulbecco's modified Eagle's medium (DMEM) containing 10% heat-inactivated fetal bovine serum.

Peptide synthesis. The Stat3 SH2 domain peptide sequence used in these experiments, FISKERERAILSTKPPGTFLLRFSESSK (SEQ ID NO:1), was purchased from Peptide 2.0 (Fairfax, Va.) at >95% purity.

Cloning and protein expression. The molecular cloning, expression, and the purification of His-tagged Stat3 and His-tagged Stat3 SH2 domain were previously described. (Zhang et al., 2010). Clones were sequenced to verify the correct sequences and/orientation. His-tagged recombinant proteins were expressed in BL21(DE3) cells, and purified on Ni-ion sepharose column.

Transient transfection of cells and treatment with SPI. 12 to 24 hours following seeding, mouse fibroblasts over-expressing hEGFR (NIH3T3/hEGFR) in 6-well plates were transiently co-transfected with 4 μg of 3-Casein Luc and 500 ng β-galactosidase (for normalizing) for 8 hours using Lipofectamine plus (Invitrogen, Carlsbad, Calif.) and following the manufacturer's protocol. 12 hours after transfection, cells were treated or untreated with increasing concentration of SPI (0-60 μM) for 12 hours prior to stimulation with rhEGF (10 ng/μL) and allowed to culture for additional 12 hours, after which cells were harvested and cytosolic extracts prepared for luciferase assay, as previously performed (Siddiquee et al, 2007; Turkson et al., 1999; Turkson et al., 1998).

Cytosolic extracts and cell lysates preparation and, luciferase assay. Cytosolic extract preparation from mammalian cells for luciferase assay are as described previously (Turkson et al., 1999; Turkson et al., 1998). Luciferase assays were carried out according to the supplier's (Promega, Madison, Wis.) manual and measured with a luminometer (Lumat LB 9507, EG&G Berthold, Germany).

Nuclear extract preparation and electrophoretic mobility shift assay. Nuclear extract preparations and electrophoretic mobility shift assay (EMSA) were carried out as previously described (Yu et al., 1995; Turkson et al., 1997). The ³²P-labeled oligonucleotide probes used were hSIE (high affinity sis-inducible element from the c-fos gene, m67 variant, 5′-AGCTTCATTTCCCGTAAATCCCTA (SEQ ID NO:19)) that binds Stat1 and Stat3 (Wagner et al., 1990) and MGFe (mammary gland factor element from the bovine (3-casein gene promoter, 5′-AGATTTCTAGGAATTCAA (SEQ ID NO:20)) for Stat1 and Stat5 binding (Gouilleux et al., 1995; Seidel et al., 1995). Where appropriate, cells in culture were pre-treated with SPI for 12 hours, prior to treatment with sodium orthovanadate for 8 hours or stimulation with EGF (10 ng/μL) for 12 minutes harvested for nuclear extract preparation.

SDS-PAGE/Western blotting analysis. SDS/PAGE and Western blotting analysis were performed as previously described (Turkson et al., 1998; Zhang et al., 2000). Primary antibodies used were anti-Stat3, pY705Stat3, pErk1/2, and Erk1/2 (Cell Signaling).

Cell viability, proliferation, and Annexin V/flow cytometry studies. Cells in culture in E-well or 96-well plates were treated with or without 50 μM SPI for 24-48 hours and subjected to CyQuant cell proliferation assay (Invitrogen Corp/Life Technologies Corp), or harvested, and the viable cells counted by trypan blue exclusion with phase contrast microscopy, or cells were processed for Annexin V and 7-AAD binding (BD Biosciences, San Jose, Calif.) with flow cytometry for apoptosis.

Fluorescence imaging and immunofluorescence with laser-scanning confocal microscopy. Studies were performed as previously reported (Jaganathan et al., 2010). Briefly, human breast cancer, MDA-MB-231 or NlH3T3/hEGFR cells were grown in multi-cell plates on slides or not, and treated with or without 5-carboxyfluorescein-labeled SPI (30 μM) for 2.5 hours. Cells were washed with 1× phosphate buffered saline (PBS), fixed with ice-cold methanol, and visualized using Zeiss Axiovert 200 microscope (Zeiss, Germany) for fluorescent images, or for confocal microscopy, cells were washed three times with 1×PBS, fixed with ice-cold methanol for 15 min, washed 3 times in PBS, permeabilized with 0.2% Triton X-100 for 10 min, and further washed 3-4 times with PBS. Specimens were then blocked in 1% bovine serum albumin (BSA) for 1 hour and incubated with anti-EGFR antibody (Santa Cruz) at 1:50 dilution at 4° C. overnight. Subsequently, cells were rinsed 4-5 times in PBS, incubated with Alexa fluor 546 rat secondary antibody for anti-EGFR antibody detection (Invitrogen) for 1 hour at room temperature in the dark. Specimens were then washed 5 times with PBS, covered with cover slides with VECTASHIELD mounting medium containing DAPI, and examined immediately under a Leica TCS SP5 confocal microscope (Germany) at appropriate wavelengths. Images were captured and processed using the Leica TCS SP5 software.

Fluorescence polarization assay. Fluorescence Polarization (FP) Assay was conducted as previously reported (Schust et al., 2006; Zhang et al., 2010) using the labeled phospho-peptide, 5-carboxyfluorescein-GpYLPQTV-NH₂ (where pY represents phospho-Tyr) as probe and Stat3 or SPI. For a saturation curves, a fixed concentration of the fluorescently-labeled peptide probe (10 nM) was incubated with increasing concentration of Stat3 (0-0.8 μM) or SPI (0-400 μM) for 30 min at room temperature in the buffer, 50 mM NaCl, 10 mM HEPES, 1 mM EDTA, 0.1% Nonidet P-40, and the fluorescent polarization measurements were determined using the POLARstar Omega (BMG LABTECH, Durham, N.C.), with the set gain adjustment at 35 mP. For evaluating the effect of SPI as an inhibitor on Stat3 binding to pY peptide, a fixed concentration of Stat3 protein (0.8 μM) was pre-incubated with serial concentrations of SPI (0-150 μM), or in the case of the effect of the Stat3 inhibitor, S3I-201 on the binding of Stat3 or SPI to probe, a fixed amount of Stat3 (200 nM) or SPI (150 μM) was pre-incubated with increasing concentration of S31-201 at 30° C. for 60 minutes in the indicated assay buffer conditions, prior to the addition of the labeled probe. Probe was then added at a final concentration of 10 nM and incubated for 30 min at room temperature following which the FP measurements were taken using the POLARstar Omega, with the set gain adjustment at 35 mP.

Surface plasmon resonance analysis. SensiQ and its analysis software Qdat (1CX Technologies, Oklahoma City, Okla.) were used to analyze the interaction between known Stat3-binding pTyr peptide motifs (analyte) and Stat3 or the Stat3 SH2 domain (target) and to determine the binding affinity, as previously reported (Zhang et al., 2010). Purified Stat3 (50 μg), Stat3 SH2 domain (30 μg), or SPI (25 μg) as was immobilized on a Carboxy Sensor Chip (for SPI) or a HisCap Sensor Chip (for Stat3 and the Stat3 SH2 domain) by injecting the peptide or protein onto the chip. Various concentrations of pTyr peptides (analyte) in running buffer (1×PBS, 0.5% DMSO) were passed over the sensor chip to produce response signals. The response signals were referenced by subtracting the response generated by passing across a surface without the analytes. The association and dissociation rate constants were calculated using the Qdat software. The ratio of the association and dissociation rate constants was determined as the affinity (K_{D}).

Statistical analysis. Statistical analysis was performed on mean values using Prism GraphPad Software, Inc. (La Jolla, Calif.). The significance of differences between groups was determined by the paired t-test at p<0.05*, <0.01**, and <0.001***.

Protein:protein interactions are a common molecular event in signal transduction and many other physiological processes. In the case of Stat3, the recruitment via the SH2 domain to cognate receptor pTyr peptide motifs is an initial step for phosphorylation. These data showed that the Stat3 SH2 domain-derived 28-mer peptide. SPI alone was sufficient to reproduce aspects of the biochemical properties of Stat3 (or the Stat3 SH2 domain), thereby acting as a motif that engaged in the inter-molecular interactions with residues of the cognate pTyr peptides to which Stat3 binds. Using biophysical analysis, such as SPR, the similarities in the binding characteristics of Stat3 (or Stat3 SH2 domain) and SPI to known cognate pTyr peptides, including the native 1L-6R/gp-130 derived peptide, GpYLPQTV-NH₂, the Stat3 peptide, pY705Stat3, and the EGFR motif, pY1068EGFR, with which Stat3 and SPI interact, and to the EGFR motif, pY1086EGFR and the Stat1 peptide, pY701Stall, which they bind to with low affinities, were all examined. The similarity in the binding characteristics of SPI and Stat3 was further evident by the SPR analysis that indicated unfavorable interactions with the native Stat5 phosphopeptide, pY695Stat5, with affinities that are in millimolar concentrations (K_{D} of 1-7 mM). SPI, like Stat3, showed preferential binding to different cognate pTyr peptide motifs, and for example, showed stronger binding to the Stat3 phosphopeptide, compared to weaker binding to the Stat1 phosphopeptide. The observed differences in the affinities indicated that the type and number of binding partners to which Stat3 (or Stat3 SH2 domain) or SPI interacted with can be influenced by their intracellular concentrations. Fluorescence polarization analysis based on the binding to the gp130-derived peptide (as 5-carboxyfluorescein-GpYLPQTV-NH₂), (Chen et al., 2010), further supported the similarities in the binding characteristics between SPI and Stat3 (data not shown).

Fluorescence polarization studies demonstrated that SPI competed against Stat3 for the binding to IL-6R/gp13-derived pTyr peptide probe. Furthermore, SPI exhibited selectivity at certain concentrations in the inhibition of intracellular Stat3 phosphorylation, DNA-binding and transcriptional activities. The inhibition of intracellular Stat3 activation may be due to the ability to disrupt Stat3:Stat3 dimerization, as has been observed for other Stat3 dimerization inhibitors (Turkson et al., 2004; Turkson et al., 2001; Siddiquee et al., 2007; Siddiquee et al., 2007), and that by associating with receptor pTyr motifs, SPI blocked Stat3-binding to receptors, and prevented de novo phosphorylation. Thus, while the treatment of cells with the protein phosphatase inhibitor, sodium orthovanadate alone, increased the levels of activated Stat3 above constitutive levels, due to the blockade of pStat3 turnover by protein phosphatases, the prior exposure of cells to SPI squelched any subsequent orthovanadate-induced accumulation of activated Stat3. Moreover, the observation that SPI exhibited preferential inhibition of Stat3 activation relative to Stat1, in spite of the physiological occurrence of a Stat 1:Stat3 heterodimer, when the two Stat family members were concurrently activated by EGF, indicated that SPI had preference for Stat3 over Stat1, but also that activated Stat3 protein can prefer to form a homodimer when concurrently activated with Stat1.

The SPI-mediated of inhibition of Stat3 activation, via binding to cognate pTyr peptide motifs, was in direct converse to the mechanisms of Stat3 inhibition by many of the existing Stat3-inhibitory modalities, which are pTyr peptide mimetics and bind to the Stat3 SH2 module (Yue et al., 2008; Turkson 2004; Turkson et al., 2004; Turkson et al., 2001; Song et al., 2005; Ren et al., 2003; Fletcher et al., 2008; Chen et al., 2010; Bhasin et al., 2008), or to the approaches that have been reported for the inhibition other SH2 domain-containing proteins, such as the adapter protein, Grb2 (Dharmawardana et al., 2006), although the biochemical outcome of the inhibition can be the same, which is to disrupt pTyr:SH2 domain interactions. While there are many protein entities with an SH2 module that is involved in promoting signal transduction and other biochemical processes (Yaffe 2002; Sawyer et al., 1998), evidence of specificity for SPI action, which was demonstrated in the lack of effect on EGF-induced Stat5 activation and transcriptional activity, Erk^{MAPK} (activation, the c-fos-promoter-driven luciferase reporter, or on Stat1 activation at concentrations that inhibit Stat3 activity.

Accordingly, antitumor cell effects of SPI were observed at concentrations that inhibit Stat3 activity and were consistent with the blockade of aberrant Stat3 activation (Yue et al., 2008; Turkson 2004; Turkson et al., 2004; Turkson et al., 2001; Song et al., 2005; Chen et al., 2010; Bhasin et al., 2008). Specifically, human breast, pancreatic, prostate, and non-small cell lung cancer cells harboring aberrant Stat3 activity were more sensitive to SPI. An increased nuclear accumulation of SPI in malignant cells was detected, which can promote inhibition of nuclear activated Stat3 and Stat3 transcriptional activity. By contrast, in mouse fibroblasts over-expressing the EGFR in which Stat3 is not aberrantly-activated, SPI is predominantly localized to the cell membrane. Herein is disclosed a Stat3 SH2 domain-mimetic that functions as an inhibitor of Stat3 activation. The approach to the inhibition of Stat3 activation by SPI avoids the known challenges of mimicking the pTyr functionality for the existing SH2 domain-binding inhibitors. Furthermore, SPI serves as a valuable molecular probe for interrogating aberrant Stat3 functions in tumor processes and for designing in vitro pTyr peptide-binding assays.

### REFERENCES

Becker et al. (1998) Nature 394, 145-151.
Bhasin et al. (2008) Bioorg Med Chem Lett 18, 391-395.
Bromberg et al. (2000) Breast Cancer Res. 2, 86-90.
Burke et al. (1999) Bioorg. Med. Chem. Lett. 9, 347-352.
Burke et al. (1994) Biochemistry 33, 6490-6494.
Chen et al. (2010) ACS Med Chem Lett 13, 85-89
Coleman et al. (2005) J. Med. Chem. 48, 6661-6670.
Darnell et al. (2005) Nat. Med. 11, 595-596.
Darnell et al. (2002) Nat. Rev. Cancer 2, 740-749.
Dharmawardana et al. (2006) Anticancer Drugs 17, 13-20.
Farr et al. (1989) Cell Immunol, 11, 427-444.
Fletcher et al. (2008) ChemMedChem, 3, 1159-1168.
Garcia et al. (2001) Oncogene 20, 2499-2513.
Gouilleux et al. (1995) Endocrinology 136, 5700-5708.
Gunning et al. (2008) Chembiochem 9, 2800-2803.
Jaganathan et al. (2010) J. Pharmacol. Exp. Ther. 333, 373-381.
Johnson et al. (1985) Mol. Cell. Biol. 5, 1073-1083.
Jones et al. (1997) J. Mol. Biol. 267, 727-748.
Pettersen et al. (2004) J. Comput. Chem. 25, 1605-1612.
Ren et al. (2003) Bioorg Med Chem Lett 13, 633-636.
Sawyer et al. (1998) Biopolymers 47, 243-261.
Schust et al. (2006) Chem Biol 13, 1235-1242.
Seidel et al. (1995) Proc. Natl. Acad. Sci. USA 92, 3041-3045.
Siddiquee et al. (2007) Proc Natl Acad Sci USA 104, 7391-7396.
Siddiquee et al. (2008) Cell Res. 18, 254-267.
Siddiquee et al. (2007) ACS Chem. Biol. 2.787-798.
Song et al. (2005) Proc Natl Acad Sci USA, 102, 4700-4705.
Turkson et al. (2004) Expert Opin Ther Targets 8, 409-422.
Turkson et al. (2000) Oncogene 19, 6613-6626.
Turkson et al., (1999) Mol. Cell. Biol. 19, 7519-7528.
Turkson et al. (1998) Mol. Cell. Biol. 18, 2545-2552.
Turkson et al. (2004) Mol Cancer Ther 3, 261-269.
Turkson et al. (2001) J. Biol, Chem, 276, 45443-45455.
Wagner et al. (1990) EMBO J, 9, 4477-4484.
Yaffe et al. (2002) Nat Rev Mol Cell Biol 3, 177-186.
Yu et al. (1995) Science 269, 81-83.
Yu et al. (2004) Nat. Rev. Cancer 4, 97-105.
Yue et al. (2008) Expert Opin Investig Drugs 18, 45-56.
Zhang et al. (2010) Biochem Pharmacol 79, 1398-1409.
Zhang et al. (2000) J. Biol. Chem. 275, 24935-24944.

1. An isolated Stat3 SH2 domain mimicking polypeptide, comprising, an amino acid sequence as set forth in SEQ ID NOs:1 or 21, or an amino acid sequence having greater than 75% homology to SEQ ID NOs:1 or 21.

2. (canceled)

3. (canceled)

4. The polypeptide of claim 1, wherein the polypeptide ammo acid sequence is FISKERERAILSTKPPGTFLLRFSESSK.

5. The polypeptide of claim 1, wherein the polypeptide ammo acid sequence is ISKERERAILSTKPP.

6. (canceled)

7. (canceled)

8. The polypeptide of claim 1, wherein the polypeptide modulates one or more of constitutive Stat3 phosphorylation, Stat3 DNA binding, Stat3 transcriptional function, or Stat3 activities in vitro or in vivo.

9. (canceled)

10. The polypeptide of claim 1, wherein the polypeptide comprises modified amino acids.

11. The polypeptide of claim 1, wherein the polypeptide is amino-terminally modified or carboxy-terminally modified, or both.

12. The polypeptide of claim 1, wherein the polypeptide is labeled.

13. (canceled)

14. (canceled)

15. The polypeptide of claim 1, wherein the polypeptide binds to receptor phosphotyrosine (pTyr) peptide motifs.

16. The polypeptide of claim 15, wherein the polypeptide inhibits binding of pTyr peptide motifs to Stat3 or Stat3 SH2 domain.

17. The polypeptide of claim 16, wherein the pTyr peptide motifs comprise native pTyr peptide, PpYLKTK, native IL-6R/gp-130 derived peptide, GpYLPQTV-NH2, the Stat3 peptide, pY705Stat3, or the EGFR motifpY1068EGFR.

18. The polypeptide of claim 15, wherein the receptor phosphotyrosine pTyr motifs comprise pTyr peptide motifs of epidermal growth factor receptor (EGFR) or pTyr peptide motifs of IL-6 receptor.

19. The polypeptide of claim 1, wherein the polypeptide modulates phosphorylation of a Stat monomer.

20. The polypeptide of claim 1, that modulates dimerization of two STAT monomers.

21. The polypeptide of claim 20, wherein the two Stat monomers are Stat3 monomers.

22. The polypeptide of claim 20, wherein at least one Stat monomer is Stat3

23. The polypeptide of claim 20, wherein at least one Stat monomer is Stat5.

24. (canceled)

25. An isolated nucleic acid encoding a Stat3 SH2 domain mimicking polypeptide comprising an amino acid sequence as set forth in SEQ ID NO:1 or SEQ ID NO:21.

26.-68. (canceled)

69. A composition, comprising, a nucleic acid encoding a Stat3 SH2 domain mimicking polypeptide comprising an amino acid sequence as set forth in SEQ ID NO:1 or SEQ ID NO:21.

70. The composition of claim 69, wherein the nucleic acid encoding a Stat3 SH2 domain mimicking polypeptide comprising an amino acid sequence as set forth in SEQ ID NO:1 or SEQ ID NO:21 is in a vector.

71. The composition of claim 69, wherein the nucleic acid encoding a Stat3 SH2 domain mimicking polypeptide comprising an amino acid sequence as set forth in SEQ ID NO:1 or SEQ ID NO:21 is in a host cell.


SEQUENCE LISTING (22 sequences)

SEQ ID NO: 1 - protein, 28 aa, Artificial Sequence
  Description of Artificial Sequence Note = Synthetic Construct
FISKERERAILSTKPPGTFLLRFSESSK

SEQ ID NO: 2 - protein, 770 aa, Artificial Sequence
  Description of Artificial Sequence Note = Synthetic Construct
MAQWNQLQQLDTRYLKQLHQLYSDTFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMDLTSECATSPM

SEQ ID NO: 3 - protein, 770 aa, Artificial Sequence
  Description of Artificial Sequence Note = Synthetic Construct
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 4 - protein, 786 aa, Artificial Sequence
  Description of Artificial Sequence Note = Synthetic Construct
MAQWNQLQQLETRYLEQLYHLYSDSFPMELRQFLAPWIESQDWAYAANKESHATLVFHNLLGEIDQQYSRFLQENNVLYQHNLRRIKQHLQSKYLEKPMEIARIVARCLWEEQRLLQTATTAQQDGQVAHPTGTVVTEKQQILEHNLQDIRKRVQDMEQKMKMLENLQDDFDFNYKTLKSAGELSQDLNGNSQAAATRQKMSQLEQMLSALDQLRRQIVTEMAGLLSAMDFVQKNLTDEELADWKRRQQIACIGGPPNICLDRLETWITSLAESQLQIRQQIRKLEELQQKVSYKGDPIIQHRPALEEKIVDLFRNLMKSAFVVERQPCMPMHPDRPLVIKTGVQFTTKVRLLVKFPELNYQLKIKVCIDKESGDVAAIRGSRKFNILGTNTKVMNMEESNNGSLSAEFKHLTLREQRCGNGGRTNSDASLIVTEELHLITFETEVYHQGLKIDLETHSLPVVVISNICQMPNAWASILWYNMLTNHPKNVNFFTKPPVGTWDQVAEVLSWQFSSTTKRGLTIEQLTTLAEKLLGPCVNYSGCQITWAKFCKENMAGKGFSFWVWLDNIIDLVKKYILALWNEGYIMGFISKERERAILSPKPPGTFLLRFSESSKEGGITFTWVEKDINGKTQIQSVEPYTKQQLNSMSFAEIIMGYKIMDATNILVSPLVYLYPDIPKEEAFGKYCRPEAHPDTEFPDTGCVTQPYLKTKFICVTPTNSGNTSDLFPMSPRTLDSLMHNEAAEANPGPLESLTLDMELSSDHASPMREGFAASTVSDMDTCRNA

SEQ ID NO: 5 - protein, 48 aa, Artificial Sequence
  Description of Artificial Sequence Note = Synthetic Construct
IIDLVKKYILALWNEGYIMGFISKERERAILSTKPPGTFLLRFSESSK

SEQ ID NO: 6 - protein, 43 aa, Artificial Sequence
  Description of Artificial Sequence Note = Synthetic Construct
KKYILALWNEGYIMGFISKERERAILSTKPPGTFLLRFSESSK

SEQ ID NO: 7 - protein, 38 aa, Artificial Sequence
  Description of Artificial Sequence Note = Synthetic Construct
ALWNEGYIMGFISKERERAILSTKPPGTFLLRFSESSK

SEQ ID NO: 8 - protein, 33 aa, Artificial Sequence
  Description of Artificial Sequence Note = Synthetic Construct
GYIMGFISKERERAILSTKPPGTFLLRFSESSK

SEQ ID NO: 9 - protein, 29 aa, Artificial Sequence
  Description of Artificial Sequence Note = Synthetic Construct
GFISKERERAILSTKPPGTFLLRFSESSK

SEQ ID NO: 10 - protein, 48 aa, Artificial Sequence
  Description of Artificial Sequence Note = Synthetic Construct
FISKERERAILSPKPPGTFLLRFSESSKEGGITFTWVEKDINGKTQIQ

SEQ ID NO: 11 - protein, 43 aa, Artificial Sequence
  Description of Artificial Sequence Note = Synthetic Construct
FISKERERAILSPKPPGTFLLRFSESSKEGGITFTWVEKDING

SEQ ID NO: 12 - protein, 38 aa, Artificial Sequence
  Description of Artificial Sequence Note = Synthetic Construct
FISKERERAILSPKPPGTFLLRFSESSKEGGITFTWVE

SEQ ID NO: 13 - protein, 33 aa, Artificial Sequence
  Description of Artificial Sequence Note = Synthetic Construct
FISKERERAILSPKPPGTFLLRFSESSKEGGIT

SEQ ID NO: 14 - protein, 29 aa, Artificial Sequence
  Description of Artificial Sequence Note = Synthetic Construct
FISKERERAILSPKPPGTFLLRFSESSKE

SEQ ID NO: 15 - protein, 48 aa, Artificial Sequence
  Description of Artificial Sequence Note = Synthetic Construct
ALWNEGYIMGFISKERERAILSTKPPGTFLLRFSESSKEGGVTFTWVE

SEQ ID NO: 16 - protein, 42 aa, Artificial Sequence
  Description of Artificial Sequence Note = Synthetic Construct
NEGYIMGFISKERERAILSTKPPGTFLLRFSESSKEGGVTFT

SEQ ID NO: 17 - protein, 38 aa, Artificial Sequence
  Description of Artificial Sequence Note = Synthetic Construct
GYIMGFISKERERAILSTKPPGTFLLRFSESSKEGGVT

SEQ ID NO: 18 - protein, 30 aa, Artificial Sequence
  Description of Artificial Sequence Note = Synthetic Construct
GFISKERERAILSTKPPGTFLLRFSESSKE

SEQ ID NO: 19 - DNA, 24 bp, Artificial Sequence
  Description of Artificial Sequence Note = Synthetic Construct
agcttcatttcccgtaaatcccta

SEQ ID NO: 20 - DNA, 18 bp, Artificial Sequence
  Description of Artificial Sequence Note = Synthetic Construct
agatttctaggaattcaa

SEQ ID NO: 21 - protein, 15 aa, Artificial Sequence
  Description of Artificial Sequence Note = Synthetic Construct
ISKERERAILSTKPP

SEQ ID NO: 22 - protein, 23 aa, Artificial Sequence
  Description of Artificial Sequence Note = Synthetic Construct
KERERAILSTKPPGTFLLRFSES